(19) United States
(12) Patent Application Publication
(10) Pub. No.: US 2025/0064805 A1
(43) Pub. Date: Feb. 27, 2025

(54) Methods of Treating Myeloproliferative Disorders Based on BTK Occupancy & Resynthesis Rate

(71) Applicant: Telios Pharma Inc., Redwood City, CA (US)

(72) Inventor: Wayne Rothbaum, New York, NY (US)

(73) Assignee: Telios Pharma Inc., Redwood City, CA (US)

(21) Appl. No.: 18/728,591

(22) PCT Filed: Jan. 13, 2023
(86) PCT No.: PCT/US2023/010805

§ 371 Date: Jul. 12, 2024

Related U.S. Application Data
(60) Provisional application No. 63/299,625, filed on Jan. 14, 2022.

(51) Int. Cl. A61K 31/506; A61P 35/02

(52) U.S. Cl. CPC A61K 31/506; A61P 35/02

(57) ABSTRACT

In an embodiment, therapeutic methods and use of a Bruton's Tyrosine Kinase (BTK) inhibitor for treatment of a myeloproliferative disorder, based on BTK occupancy and/or BTK resynthesis rates in various tissue compartments, including blood, spleen bone marrow, liver and lymph nodes are described.

### FIELD OF THE INVENTION

Treatment of myeloproliferative disorders with Bruton's tyrosine kinase (BTK) inhibitors based on BTK occupancy and/or resynthesis rate are disclosed herein.

### BACKGROUND OF THE INVENTION

Myelofibrosis (MF) is a chronic leukemia, a cancer that affects the blood-forming tissues in the body. Myelofibrosis belongs to a group of diseases called myeloproliferative disorders and is an uncommon type of bone marrow cancer that disrupts the normal production of blood cells. Myelofibrosis causes extensive fibrosis in bone marrow and dysfunction of normal hematopoiesis, leading to severe anemia that can cause weakness and fatigue and it can also cause a low number of platelets, which increases the risk of bleeding. Myelofibrosis often causes an enlarged spleen and lymph nodes due extramedullary hematopoiesis and the accumulation of malignant myeloid cells in the spleen. Additionally, patients frequently suffer from constitutional symptoms (e.g., fatigue, night sweats, weight loss, pruritus, fever, bone and joint pain) related to aberrant overproduction of pro-inflammatory cytokines.

The clinical spectrum of MF includes primary myelofibrosis and MF that develops during essential thrombocythemia or polycythemia vera. Myelofibrosis is a chronic hematologic malignancy characterized by splenomegaly, leukoerythroblastosis, cytopenias, teardrop poikilocytosis, marrow fibrosis, extramedullary hematopoiesis, increased marrow microvessel density, and constitutive mobilization of CD34+ hematopoietic stem cells (HSC) and progenitor cells (HPC).

Myelofibrosis is also characterized by abnormal trafficking and homing of HSC and HPC in the bone marrow and peripheral blood, resulting in their constitutive mobilization and the establishment of splenomegaly. Splenomegaly often is associated with an increased plasma volume and an increased whole blood volume, both of which may correlate with the degree of splenic enlargement (Pengelly (1977) J R Coll Physicians Lond. 12, 61-66). CXCR4-CXCL12 (CXCL12 also known as SDF-1) signaling plays a critical role in a variety of processes underlying proper lymphoid and myeloid cell development and function, including development and retention of precursor cells in the bone marrow, homing of immature and mature cells to secondary lymphoid organs and trafficking and homing of plasma cells to the bone marrow. In MF, the constitutive mobilization of HSC and HPC has been associated with profound alterations in the CXCR4-CXCL12 axis, which occur because of downregulation of CXCR4 expression by myelofibrotic cells and/or CD34+ cells due to hypermethylation of the CXCR4 promoter, and the proteolytic degradation of CXCL12. In the spleen of MF patients, CXCL12 and integrins such as Very Late Antigen-4 (VLA-4) are highly expressed and CXCL12 acts as a chemo-attractant for the mobilized CD34+ cells. This contrasts with the bone marrow and peripheral blood, where CXCL12 expression levels are abnormally low. Drawn to the spleen via CXCL12, CD34+ cells interact with stromal cells through adhesion molecules such as VLA-4 and its ligand VCAM-1 (vascular cell adhesion molecule 1), potentiating extramedullary hematopoiesis and splenomegaly. This aberrant stem cell behavior can be influenced, not only by intrinsic properties of the stem cells, but also by regulatory signals provided by the MF microenvironment (Wang (2015) Experimental Hematology 43, 100-109). Therefore, the ability to manipulate cell trafficking, homing and sequestering via these pathways represents an opportunity to treat MF patients.

Bruton's Tyrosine Kinase is a non-receptor tyrosine kinase that belongs to the Tec family and has an important function in certain benign and malignant cells of the hematopoietic system. Moreover, recent clinical studies with irreversible oral BTK inhibitors, acalabrutinib and ibrutinib, have demonstrated excellent clinical activity and tolerability against a variety of B-cell malignancies including: chronic lymphocytic leukemia (CLL), mantle cell lymphoma (MCL), Waldenstrom macroglobulinemia (WM), marginal zone lymphoma (MZL) and diffuse large B-cell lymphoma (DLBCL). Furthermore, it is now clear the mechanism of action of BTK inhibitors is multifactorial, with a significant component of its function involving the disruption of the tumor cell with the microenvironment that protects it. Inhibition of BTK has been shown to alter malignant B-cell migration in CLL and MCL, and malignant myeloid cell migration in acute myeloid leukemia (AML), by inhibiting CXCR4-CXCL12 induced cell trafficking, homing and cell surface adhesion through reduced expression of numerous vascular adhesion molecules (Zaitseva (2014) Oncotarget 5, 9930-9938) including alpha-beta integrins. CXCL12 plays a central role in CLL pathogenesis and progression, by supporting CLL cell-to-cell interaction with the stromal microenvironment, leading to re-enforcement of cell-survival and proliferative signalling. BTK has a key role in signal transduction, between the CXCR4-CXCL12 signaling axis, and activity of cell-surface integrins. BTK inhibition prevents CXCL12-induced activation of lymphocyte function-associated antigen-1 (LFA-1) and VLA-4 alpha-beta integrins. Furthermore, BTK inhibition blocks the activation of the small GTP-binding protein RhoA, controlling integrin affinity. Very importantly, BTK-tyrosine-phosphorylation and activation by CXCL12 depends on upstream activation of JAK2 (Janus kinase 2). Thus, BTK and JAK protein tyrosine kinases manifest a hierarchical activity both in chemokine and integrin activation and dependent cell adhesion (Montresor (2018) Oncotarget, 9, 35123-35140). Notably, a mutation in JAK2 (JAK2-V617F) results in constitutive kinase activity controlling the intracellular signaling of VLA-4 and LFA-1 integrins in leukocytes (Edelmann (2018) J Clin Invest. 128, 4359-4371). Lastly, BTK is highly expressed on both mature and primitive myeloid cells; including HSC and HPC. The CXCR4-CXCL12 signaling axis is a critical means of mobilization and homing for CD34+ cells.

Regulation of BTK expression levels during myeloid cell activation is tightly controlled; in hematopoiesis and in pre-and pro-B cell stages, the BTK level is relatively high. In peripheral tissues, resting B lymphocytes have lower BTK than is observed in bone marrow. Stimulation of BCR leads to rapid induction of BTK expression, with increases of 10-fold protein levels within several hours of stimulation, as described in (Nisitani, PNAS. 2000, 97, 2737-2742). In B cells, expression of BTK results from NFκB-mediated transcriptional activation in addition to a post-translational mechanism that occurs rapidly after BCR stimulation (Yu, Blood, 2008, 111(9), 4617-4626). Since BTK signaling induces NFκB, there is a positive feedback loop in activated B cells. These findings suggest that inhibition of BTK affects both the downstream activity of BCR engagement, such as antibody production, as well as inhibiting the expression of BTK itself, which could further regulate the reactivity of autoimmune B cells.

The present invention includes the unexpected discovery that the rate of BTK resynthesis per malignant myeloid cell and the rate of regeneration of BTK-expressing myeloid cells following treatment of a human with a covalent inhibitor of BTK, differs from that previously identified in B-cells from patients with B-cell hematological cancers, and also from normal human subjects, and can also differ between individuals that are otherwise affected by the same myeloproliferative disorder.

Additionally, the present invention includes the novel finding that in humans, treatment with a covalent BTK inhibitor directly impacts the resynthesis rate of BTK in myeloid cells, causing a decrease in BTK resynthesis rate once full inhibition has been attained and leading to reduced BTK expression on a per-myeloid cell basis in human subjects suffering from myeloproliferative neoplasms.

Additionally, different compartments within the body have different BTK resynthesis rates. In an embodiment, the method of treating myeloproliferative disorders with a BTK inhibitor relates to treating the BTK resynthesis compartment for this disorder, in effect tailoring the dosing regimen of a BTK inhibitor to resynthesis rate in that compartment.

### SUMMARY OF THE INVENTION

The disclosure relates to a method of treating a myeloproliferative disorder in a human subject in need thereof comprising: (a) administering a Bruton's tyrosine kinase (BTK) inhibitor or a pharmaceutically acceptable salt thereof to the human at a first dose for a first period of time sufficient to provide 95% or greater BTK occupancy in a tissue compartment; and (b) administering the BTK inhibitor or a pharmaceutically acceptable salt thereof to the human at a second dose for a second period of time, wherein the second dose is equal to, or less than the first dose, and is sufficient to maintain the 95% or greater BTK occupancy in the tissue compartment. In some embodiments, the target BTK occupancy is selected from the group consisting of greater than 96%, greater than 97%, greater than 98%, and greater than 99%. In some embodiments, the BTK occupancy is estimated by the BTK resynthesis rate in a tissue compartment containing malignant myeloid cells. In some embodiments, the tissue compartment comprises hematopoietic tissue. In some embodiments, the hematopoietic tissue is extramedullary hematopoietic tissue. In some embodiments, the tissue compartment is selected from the group consisting of peripheral blood, bone marrow, lymph node, liver and spleen. In some embodiments, the BTK occupancy is evaluated based on the average BTK resynthesis rate in a population of patients with a myeloid cell signaling disorder.

In some embodiments, the method further comprises the step of determining the target BTK occupancy in the tissue compartment using a relative resynthesis rate.

In some embodiments, the BTK inhibitor is selected from Table 1. In some embodiments, the BTK inhibitor is

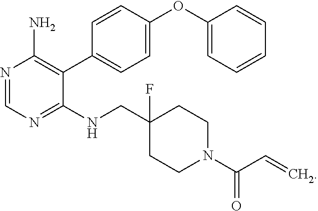 [SMILES: C=CC(=O)N1CCC(F)(CNc2ncnc(N)c2-c2ccc(Oc3ccccc3)cc2)CC1]

In some embodiments, the BTK inhibitor is administered orally. In some embodiments, the first dose is 150 mg of the BTK inhibitor. In some embodiments, the second dose is 150 mg of the BTK inhibitor.

In some embodiments, the myeloproliferative disorder is a mononuclear myeloid cell malignancy. In some embodiments, the myeloproliferative disorder is a polymorphonuclear myeloid cell malignancy. In some embodiments, the myeloproliferative disorder is a megakaryocyte malignancy, including a megakaryocyte progenitor cell malignancy. In some embodiments, the myeloproliferative disorder is an erythroid progenitor cell malignancy, including a malignancy which effects erythroid progenitor cells. In some embodiments, the myeloproliferative disorder is primary myelofibrosis, secondary myelofibrosis, myelofibrosis secondary to polycythemia vera, myelofibrosis secondary to essential thrombocythemia, myelofibrosis secondary to chronic myeloid leukemia, or idiopathic myelofibrosis.

In some embodiments, the BTK inhibitor has a serum half-life of 2.5 hours or less. In some embodiments, the first dose of the BTK inhibitor is administered once daily. In some embodiments, the first dose of the BTK inhibitor is administered twice daily. In some embodiments, the first dose of the BTK inhibitor is administered three times daily. In some embodiments, the second dose of the BTK inhibitor is administered once daily. In some embodiments, the second dose of the BTK inhibitor is administered twice daily. In some embodiments, the second dose of the BTK inhibitor is administered three times daily. In some embodiments, the first dose of the BTK inhibitor is selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, and 450 mg. In some embodiments, the second dose of the BTK inhibitor is selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, and 450 mg. In some embodiments, the first period is selected from the group consisting of 1 day, 2days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, and 21 days. In some embodiments, the second period is selected from the group consisting of 2 weeks, 1 month, 2 months, 3 months, 6 months, 9months, and 1 year.

### BRIEF DESCRIPTION OF THE DRAWINGS

The foregoing summary, as well as the following detailed description of the invention, will be better understood when read in conjunction with the appended drawings.

FIG. 1 illustrates that BTK resynthesis is faster in myeloid cells versus B cells.

FIG. 2 illustrates BTK resynthesis rate in myeloid cells over time.

FIG. 3 illustrates BTK resynthesis rate in AML and MF cells over time.

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs. All patents and publications referred to herein are incorporated by reference in their entireties.

The terms “administered in combination with” and “co-administration” as used herein, encompass administration of two or more active pharmaceutical ingredients to a subject so that both agents and/or their metabolites are present in the subject at the same time. Co-administration includes simultaneous administration in separate compositions, administration at different times in separate compositions, or administration in a composition in which two or more agents are present.

The term “effective amount” or “therapeutically effective amount” or “amount sufficient” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g. malignant myeloid cells or CD34+ cells). The specific dose will vary depending on the particular compounds chosen, the dosing regimen to be followed, whether the compound is administered in combination with other compounds, timing of administration, the tissue to which it is administered, and the physical delivery system in which the compound is carried.

“Myelofibrosis” refers to chronic and/or progressive scarring (fibrosis) of the bone marrow that disrupts the normal production of blood cells, leading to severe anemia and enlargement of the spleen, lymph nodes and liver. It can be associated with a variety of diseases, primarily myeloproliferative (preleukemic) disorders. It is also known as agnogenic myeloid metaplasia. Myelofibrosis, as used herein, includes but is not limited to, primary myelofibrosis, post-polycythemia vera myelofibrosis, and post-essential thrombocythemia myelofibrosis. Myelofibrosis as used herein, is characterized by accumulation of malignant myeloid cells in the bone marrow, spleen (i.e. splenomegaly) and lymph nodes, some of these malignant myeloid cells being CD34+. As used herein, “myeloproliferative disorder” is synonymous with “myelofibrosis” throughout the specification and claims.

The term “splenomegaly” as used herein refers to an enlargement of the spleen, measured by size or weight. In some embodiments, the enlargement is due to sequestration of malignant CD34+ myeloid cells and the resulting extramedullary hematopoiesis which develops.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the invention is contemplated. Supplementary active ingredients can also be incorporated into the described compositions.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

The BTK inhibitor may be any BTK inhibitor known in the art. BTK inhibitor compounds of the invention also include crystalline and amorphous forms of the any of the compounds in Table 1, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to. In some embodiments, the BTK inhibitor is a covalent BTK inhibitor which irreversibly binds to BTK.

TABLE 1
BTK Inhibitors

| No. | IUPAC Name | Structure |
|---|---|---|
| 1. | Acalabrutinib ((S)-4-(8-amino-3-(1-(but-2- ynoyl)pyrrolidin-2-yl)imidazo[1,5-a]pyrazin- 1-yl)-N-(pyridin-2-yl)benzamide) | 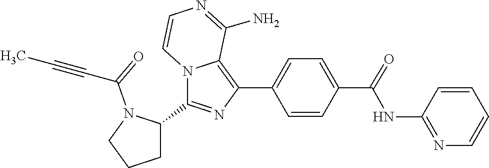 [SMILES: CC#CC(=O)N1CCC[C@H]1c1nc(-c2ccc(C(=O)Nc3ccccn3)cc2)c2c(N)nccn12] |
| 2. | Ibrutinib (1-[(3R)-3-[4-amino-3-(4- phenoxyphenyl)pyrazolo[3,4-d]pyrimidin-1- yl]piperidin-1-yl]prop-2-en-1-one) | 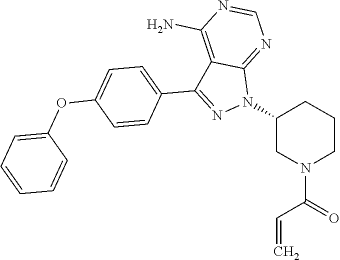 [SMILES: C=CC(=O)N1CCC[C@@H](n2nc(-c3ccc(Oc4ccccc4)cc3)c3c(N)ncnc32)C1] |
| 3. | (7S)-2-(4-phenoxyphenyl)-7-(1-prop-2- enoylpiperidin-4-yl)-4,5,6,7- tetrahydropyrazolo[1,5-a]pyrimidine-3- carboxamide | 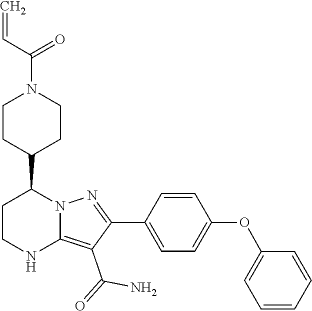 [SMILES: C=CC(=O)N1CCC([C@@H]2CCNc3c(C(N)=O)c(-c4ccc(Oc5ccccc5)cc4)nn32)CC1] |
| 4. | 2-(4-phenoxyphenyl)-7-(1-prop-2- enoylpiperidin-4-yl)-4,5,6,7- tetrahydropyrazolo[1,5-a]pyrimidine-3- carboxamide | 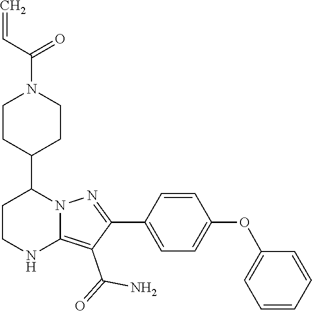 [SMILES: C=CC(=O)N1CCC(C2CCNc3c(C(N)=O)c(-c4ccc(Oc5ccccc5)cc4)nn32)CC1] |
| 5. | (7R)-2-(4-phenoxyphenyl)-7-(1-prop-2- enoylpiperidin-4-yl)-4,5,6,7- tetrahydropyrazolo[1,5-a]pyrimidine-3- carboxamide | 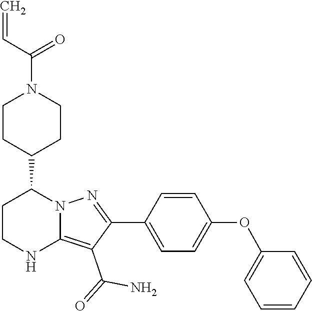 [SMILES: C=CC(=O)N1CCC([C@H]2CCNc3c(C(N)=O)c(-c4ccc(Oc5ccccc5)cc4)nn32)CC1] |
| 6. | 6-amino-9-[(3R)-1-but-2-ynoylpyrrolidin-3- yl]-7-(4-phenoxyphenyl)purin-8-one | 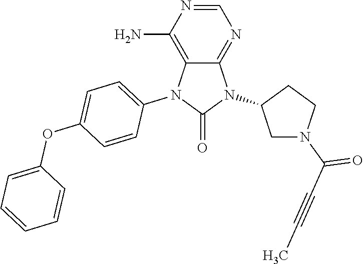 [SMILES: CC#CC(=O)N1CC[C@@H](n2c(=O)n(-c3ccc(Oc4ccccc4)cc3)c3c(N)ncnc32)C1] |
| 7. | N-[3-[5-fluoro-2-[4-(2- methoxyethoxy)anilino]pyrimidin-4- yl]amino]phenyl]prop-2-enamide | 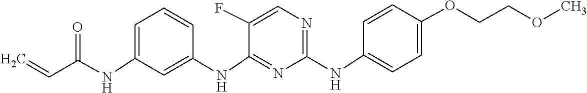 [SMILES: C=CC(=O)Nc1cccc(Nc2nc(Nc3ccc(OCCOC)cc3)ncc2F)c1] |
| 8. | Fenebrutinib (10-[3-(hydroxymethyl)-4-[1- methyl-5-[[5-[(2S)-2-methyl-4-(oxetan-3- yl)piperazin-1-yl]pyridin-2-yl]amino]-6- oxopyridin-3-yl]pyridin-2-yl]-4,4-dimethyl- 1,10-diazatricyclo[6.4.0.0^{2,6}]dodeca-2(6),7- dien-9-one) | 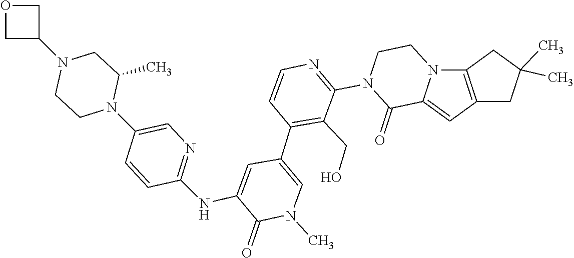 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1] |
| 9. | 1-[4-[[[6-amino-5-(4- phenoxyphenyl)pyrimidin-4- yl]amino]methyl]piperidin-1-yl]prop-2-en- 1-one | 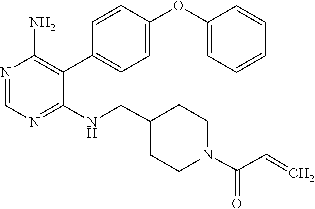 [SMILES: C=CC(=O)N1CCC(CNc2ncnc(N)c2-c2ccc(Oc3ccccc3)cc2)CC1] |
| 10. | 1-[4-[[[6-amino-5-(4- phenoxyphenyl)pyrimidin-4- yl]amino]methyl]piperidin-1-yl]prop-2-en- 1-one | 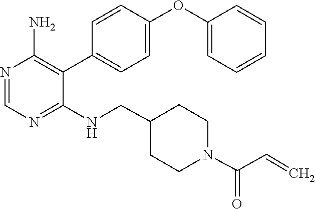 [SMILES: C=CC(=O)N1CCC(CNc2ncnc(N)c2-c2ccc(Oc3ccccc3)cc2)CC1] |
| 11. | (2-chloro-4-phenoxyphenyl)-[4-[[(3R,6S)-6- (hydroxymethyl)oxan-3-yl]amino]-7H- pyrrolo[2,3-d]pyrimidin-5-yl]methanone | 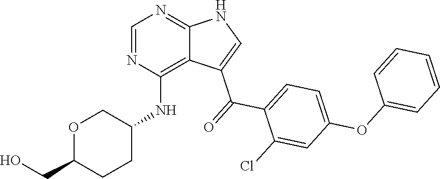 [SMILES: O=C(c1ccc(Oc2ccccc2)cc1Cl)c1c[nH]c2ncnc(N[C@@H]3CC[C@@H](CO)OC3)c12] |
| 12. | N-[3-[6-[4-[(2R)-1,4-dimethyl-3- oxopiperazin-2-yl]anilino]-4-methyl-5- oxopyrazin-2-yl]-2-methylphenyl]-4,5,6,7- tetrahydro-1-benzothiophene-2- carboxamide | 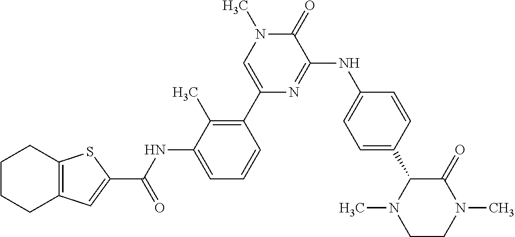 [SMILES: Cc1c(NC(=O)c2cc3c(s2)CCCC3)cccc1-c1cn(C)c(=O)c(Nc2ccc([C@@H]3C(=O)N(C)CCN3C)cc2)n1] |
| 13. | 2-[2-[2-[4-[4-amino-3-(4- phenoxyphenyl)pyrazolo[3,4-d]pyrimidin-1- yl]piperidin-1-yl]ethoxy]ethoxy]-N-[2-(2,6- dioxopiperidin-3-yl)-1,3-dioxoisoindol-5- yl]acetamide | 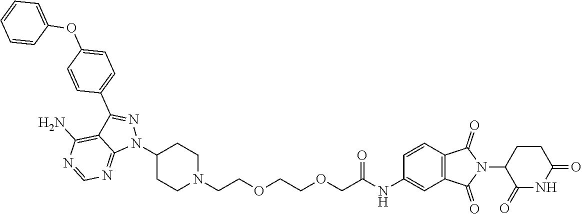 [SMILES: Nc1ncnc2c1c(-c1ccc(Oc3ccccc3)cc1)nn2C1CCN(CCOCCOCC(=O)Nc2ccc3c(c2)C(=O)N(C2CCC(=O)NC2=O)C3=O)CC1] |
| 14. | N-[3-[2-[4-(4-methylpiperazin-1- yl)anilino]furo[3,2-d]pyrimidin-4- yl]oxyphenyl]prop-2-enamide | 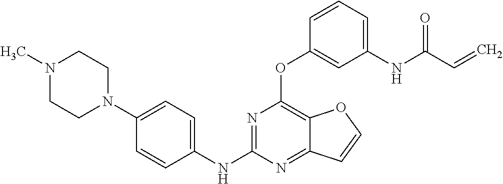 [SMILES: C=CC(=O)Nc1cccc(Oc2nc(Nc3ccc(N4CCN(C)CC4)cc3)nc3ccoc23)c1] |
| 15. | 4-tert-butyl-N-[2-methyl-3-[1-methyl-5-[4- (morpholine-4-carbonyl)-3-(prop-2- enoylamino)anilino]-6-oxopyridin-3- yl]phenyl]benzamide | 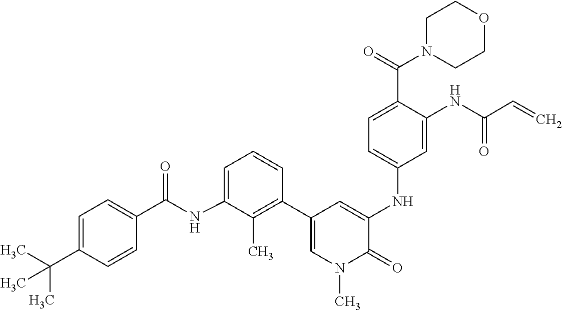 [SMILES: C=CC(=O)Nc1cc(Nc2cc(-c3cccc(NC(=O)c4ccc(C(C)(C)C)cc4)c3C)cn(C)c2=O)ccc1C(=O)N1CCOCC1] |
| 16. | (R,E)-2-(3-(4-amino-3-(2-fluoro-4- phenoxyphenyl)-1H-pyrazolo[3,4- d]pyrimidin-1-yl)piperidine-1-carbonyl)-4- methyl-4-(4-(oxetan-3-yl)piperazin-1- yl)pent-2-enenitrile | 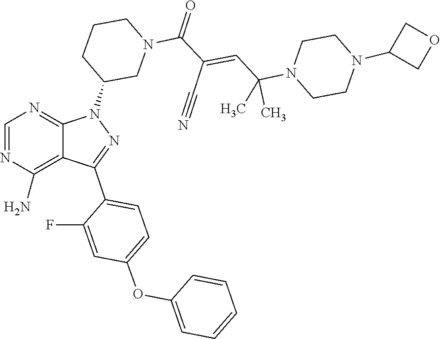 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1] |
| 17. | Branebrutinib ((S)-4-(3-(but-2- ynamido)piperidin-1-yl)-5-fluoro-2,3- dimethyl-1H-indole-7-carboxamide) | 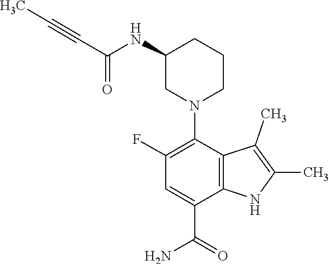 [SMILES: CC#CC(=O)N[C@H]1CCCN(c2c(F)cc(C(N)=O)c3[nH]c(C)c(C)c23)C1] |
| 18. | 4-(tert-Butyl)-N-(2-methyl-3-(4-methyl-6- ((4-(morpholine-4-carbonyl)phenyl)amino)- 5-oxo-4,5-dihydropyrazin-2- yl)phenyl)benzamide | 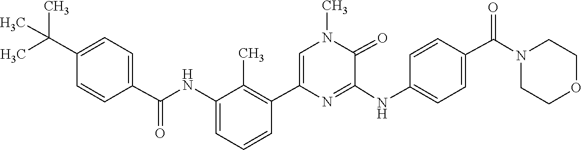 [SMILES: Cc1c(NC(=O)c2ccc(C(C)(C)C)cc2)cccc1-c1cn(C)c(=O)c(Nc2ccc(C(=O)N3CCOCC3)cc2)n1] |
| 19. | N-(1-(7H-Pyrrolo[2,3-d] pyrimidin-4- yl)piperidin-3-yl)-2-((3-chlorophenyl)amino) acetamide | 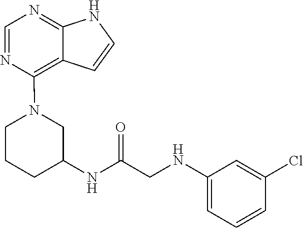 [SMILES: O=C(CNc1cccc(Cl)c1)NC1CCCN(c2ncnc3[nH]ccc23)C1] |
| 20. | 6-cyclopropyl-8-fluoro-2-[2- (hydroxymethyl)-3-[1-methyl-5-[[5-(4- methylpiperazin-1-yl)pyridin-2-yl]amino]-6- oxopyridin-3-yl]phenyl]isoquinolin-1-one | 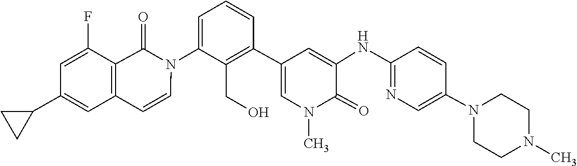 [SMILES: CN1CCN(c2ccc(Nc3cc(-c4cccc(-n5ccc6cc(C7CC7)cc(F)c6c5=O)c4CO)cn(C)c3=O)nc2)CC1] |
| 21. | N-[5-[9-[4-(methanesulfonamido)phenyl]- 2-oxobenzo[h][1,6]naphthyridin-1-yl]-2- methylphenyl]prop-2-enamide | 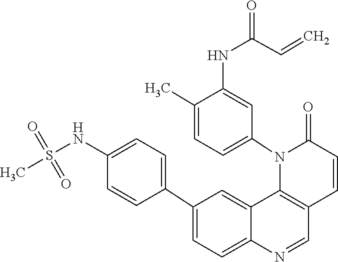 [SMILES: C=CC(=O)Nc1cc(-n2c(=O)ccc3cnc4ccc(-c5ccc(NS(C)(=O)=O)cc5)cc4c32)ccc1C] |
| 22. | 4-(4-((4-((3-acrylamidophenyl)amino)-5- fluoropyrimidin-2-yl)amino)phenoxy)-N- methylpicolinamide | 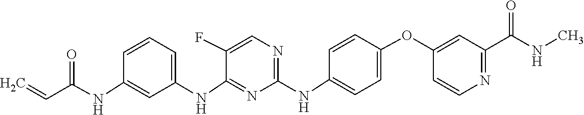 [SMILES: C=CC(=O)Nc1cccc(Nc2nc(Nc3ccc(Oc4ccnc(C(=O)NC)c4)cc3)ncc2F)c1] |
| 23. | (7S)-3-fluoro-4-[3-(8-fluoro-1-methyl-2,4- dioxoquinazolin-3-yl)-2-methylphenyl]-7-(2- hydroxypropan-2-yl)-6,7,8,9-tetrahydro-5H- carbazole-1-carboxamide | 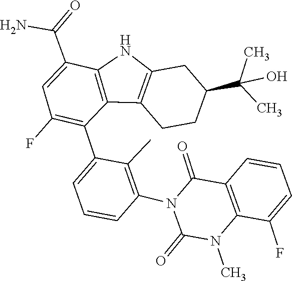 [SMILES: Cc1c(-c2c(F)cc(C(N)=O)c3[nH]c4c(c23)CC[C@H](C(C)(C)O)C4)cccc1-n1c(=O)c2cccc(F)c2n(C)c1=O] |
| 24. | 1-[3-fluoro-4-[7-(5-methyl-1H-imidazol-2- yl)-1-oxo-2,3-dihydroisoindol-4-yl]phenyl]- 3-[3-(trifluoromethyl)phenyl]urea | 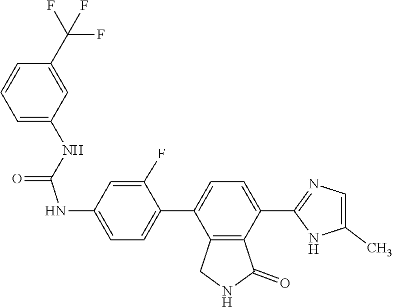 [SMILES: Cc1cnc(-c2ccc(-c3ccc(NC(=O)Nc4cccc(C(F)(F)F)c4)cc3F)c3c2C(=O)NC3)[nH]1] |
| 25. | 9-(1-methylpyrazol-4-yl)-1-(1-prop-2-enoyl- 2,3-dihydroindol-6- yl)benzo[h][1,6]naphthyridin-2-one | 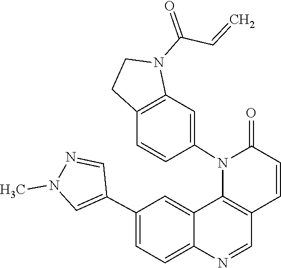 [SMILES: C=CC(=O)N1CCc2ccc(-n3c(=O)ccc4cnc5ccc(-c6cnn(C)c6)cc5c43)cc21] |
| 26. | 7-(2-hydroxypropan-2-yl)-4-[2-methyl-3-(4- oxoquinazolin-3-yl)phenyl]-9H-carbazole-1- carboxamide | 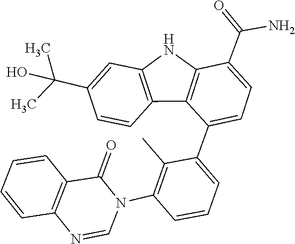 [SMILES: Cc1c(-c2ccc(C(N)=O)c3[nH]c4cc(C(C)(C)O)ccc4c23)cccc1-n1cnc2ccccc2c1=O] |
| 27. | 10-[2-(Hydroxymethyl)-3-[1-methyl-6-oxo- 5-(pyrimidin-4-ylamino)pyridin-3- yl]phenyl]-4,4-dimethyl-7-thia-10- azatricyclo[6.4.0.0^{2,6}]dodeca-1(8),2(6)- dien-9-one | 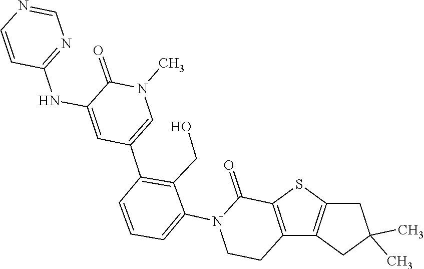 [SMILES: Cn1cc(-c2cccc(N3CCc4c(sc5c4CC(C)(C)C5)C3=O)c2CO)cc(Nc2ccncn2)c1=O] |
| 28. | (S)-5-amino-1-(1-cyanopiperidin-3-yl)-3-(4- (2,4-difluorophenoxy)phenyl)-1H-pyrazole- 4-carboxamide | 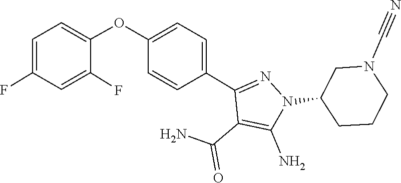 [SMILES: N#CN1CCC[C@H](n2nc(-c3ccc(Oc4ccc(F)cc4F)cc3)c(C(N)=O)c2N)C1] |
| 29. | (S)-4-(3-(1-Acryloylpyrrolidin-2-yl)-8- aminoimidazo[1,5-a] pyrazin-1-yl)-N- (pyridin-2-yl)benzamide | 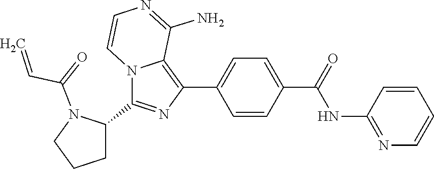 [SMILES: C=CC(=O)N1CCC[C@H]1c1nc(-c2ccc(C(=O)Nc3ccccn3)cc2)c2c(N)nccn12] |
| 30. | (S,E)-4-(8-Amino-3-(1-(4- (dimethylamino)but-2-enoyl)pyrrolidin-2- yl)imidazo[1,5-a]pyrazin-1-yl)-N(pyridin-2- yl)benzamide | 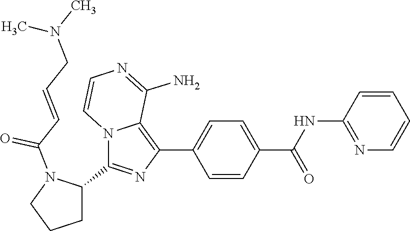 [SMILES: CN(C)C/C=C/C(=O)N1CCC[C@H]1c1nc(-c2ccc(C(=O)Nc3ccccn3)cc2)c2c(N)nccn12] |
| 31. | (S)-4-(8-Amino-3-(1-but-2-ynoylpyrrolidin- 2-yl)imidazo[1,5-a]pyrazin-1-yl)-N-(4- methylpyridin-2-yl)benzamide | 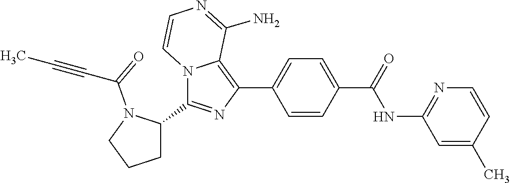 [SMILES: CC#CC(=O)N1CCC[C@H]1c1nc(-c2ccc(C(=O)Nc3cc(C)ccn3)cc2)c2c(N)nccn12] |
| 32. | (S,E)-4-(8-Amino-3-(1-(4-methoxybut-2- enoyl)pyrrolidin-2-yl)imidazo[1,5-a]pyrazin- 1-yl)-N-(4-propylpyridin-2-yl)benzamide | 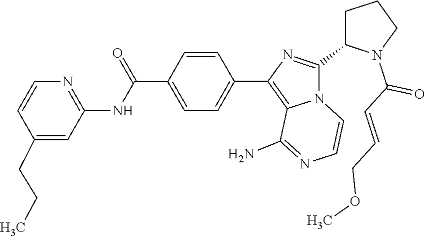 [SMILES: CCCc1ccnc(NC(=O)c2ccc(-c3nc([C@@H]4CCCN4C(=O)/C=C/COC)n4ccnc(N)c34)cc2)c1] |
| 33. | (S)-4-(8-Amino-3-(1-but-2-ynoylpyrrolidin- 2-yl)imidazo[1,5-a] pyrazin-1-yl)-N-(4- (trifluoromethyl)pyridin-2- yl)benzamide | 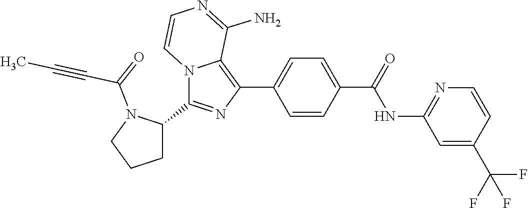 [SMILES: CC#CC(=O)N1CCC[C@H]1c1nc(-c2ccc(C(=O)Nc3cc(C(F)(F)F)ccn3)cc2)c2c(N)nccn12] |
| 34. | (S)-4-(8-Amino-3-(1-but-2-ynoylpyrrolidin- 2-yl)imidazo[1,5-a]pyrazin-1-yl)-N-(4,5,6,7- tetrahydrobenzo[d]thiazol-2-yl)benzamide | 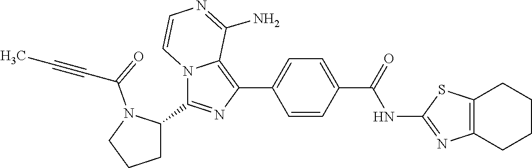 [SMILES: CC#CC(=O)N1CCC[C@H]1c1nc(-c2ccc(C(=O)Nc3nc4c(s3)CCCC4)cc2)c2c(N)nccn12] |
| 35. | (S)-4-(3-(1-acryloylpyrrolidin-2-yl)-8- aminoimidazo[1,5-a] pyrazin-1-yl)-2-fluoro- N-(pyridin-2-yl)benzamide | 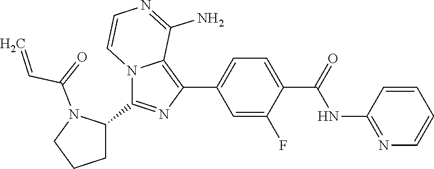 [SMILES: C=CC(=O)N1CCC[C@H]1c1nc(-c2ccc(C(=O)Nc3ccccn3)c(F)c2)c2c(N)nccn12] |
| 36. | (S)-4-(3-(1-Acryloylpyrrolidin-2-yl)-8- aminoimidazo[1,5-a]pyrazin-1-yl)-2- methoxy-N-(pyridin-2- yl)benzamide | 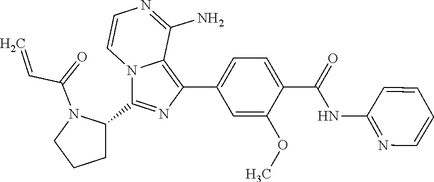 [SMILES: C=CC(=O)N1CCC[C@H]1c1nc(-c2ccc(C(=O)Nc3ccccn3)c(OC)c2)c2c(N)nccn12] |
| 37. | (S, E)-4-(8-Amino-3-(1-(4- (dimethylamino)but-2-enoyl)pyrrolidin-2- yl)imidazo[1,5-a]pyrazin-1-yl)-N-(thiazol- 2- yl)benzamide | 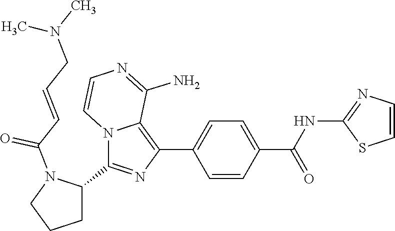 [SMILES: CN(C)C/C=C/C(=O)N1CCC[C@H]1c1nc(-c2ccc(C(=O)Nc3nccs3)cc2)c2c(N)nccn12] |
| 38. | (S)-4-(3-(1-Acryloylpiperidin-2-yl)-8- aminoimidazo[1,5-a] pyrazin-1-yl)-N-(4- fluoropyridin-2-yl)benzamide | 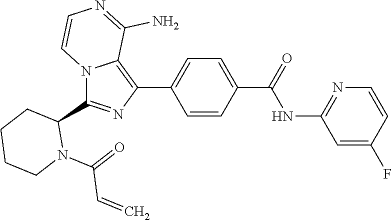 [SMILES: C=CC(=O)N1CCCC[C@H]1c1nc(-c2ccc(C(=O)Nc3cc(F)ccn3)cc2)c2c(N)nccn12] |
| 39. | (S)-4-(3-(1-Acryloylpiperidin-2-yl)-8- aminoimidazo[1,5-a] pyrazin-1-yl)-N-(4- cyanopyridin-2-yl)benzamide | 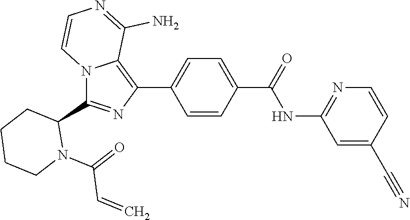 [SMILES: C=CC(=O)N1CCCC[C@H]1c1nc(-c2ccc(C(=O)Nc3cc(C#N)ccn3)cc2)c2c(N)nccn12] |
| 40. | (S)-4-(8-Amino-3-(1-(vinylsulfonyl)piperidin- 2-yl)imidazo[1,5-a] pyrazin-1-yl)-N-(4- (trifluoromethyl)pyridin-2- yl)benzamide | 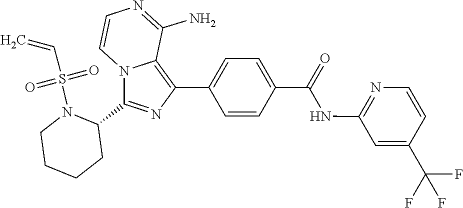 [SMILES: C=CS(=O)(=O)N1CCCC[C@H]1c1nc(-c2ccc(C(=O)Nc3cc(C(F)(F)F)ccn3)cc2)c2c(N)nccn12] |
| 41. | (S)-4-(3-(1-Acryloylpiperidin-2-yl)-8- aminoimidazo[1,5-a] pyrazin-1-yl)-N- (pyrimidin-2-yl)benzamide | 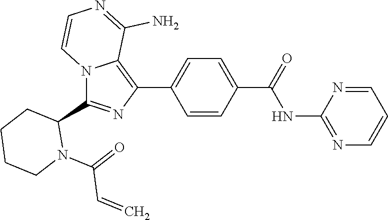 [SMILES: C=CC(=O)N1CCCC[C@H]1c1nc(-c2ccc(C(=O)Nc3ncccn3)cc2)c2c(N)nccn12] |
| 42. | (S)-4-(3-(1-Acryloylpiperidin-2-yl)-8- aminoimidazo[1,5-a] pyrazin-1-yl)-N-(4- methylpyrimidin-2-yl)benzamide | 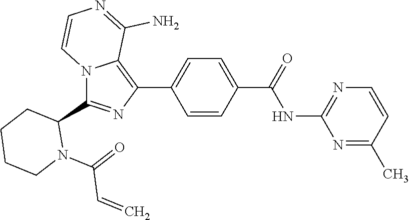 [SMILES: C=CC(=O)N1CCCC[C@H]1c1nc(-c2ccc(C(=O)Nc3nccc(C)n3)cc2)c2c(N)nccn12] |
| 43. | (S)-4-(8-Amino-3-(1-but-2-ynoylpiperidin-2- yl)imidazo[1,5-a] pyrazin-1-yl)-N- (pyrimidin-4-yl)benzamide | 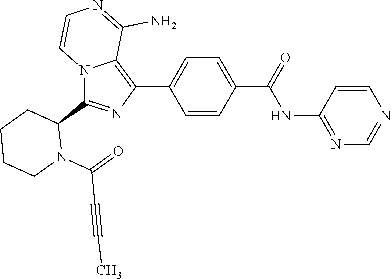 [SMILES: CC#CC(=O)N1CCCC[C@H]1c1nc(-c2ccc(C(=O)Nc3ccncn3)cc2)c2c(N)nccn12] |
| 44. | (S)-4-(8-Amino-3-(1-but-2-ynoylpiperidin-2- yl)imidazo[1,5-a] pyrazin-1-yl)-N-(pyridazin- 3-yl)benzamide | 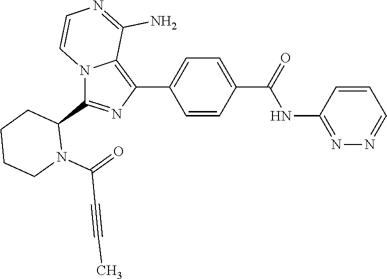 [SMILES: CC#CC(=O)N1CCCC[C@H]1c1nc(-c2ccc(C(=O)Nc3cccnn3)cc2)c2c(N)nccn12] |
| 45. | (S, E)-4-(8-Amino-3-(1-(4-methoxybut-2- enoyl)piperidin-2-yl)imidazo[1,5-a] pyrazin-1-yl)-N-(5-ethylthiazol-2- yl)benzamide | 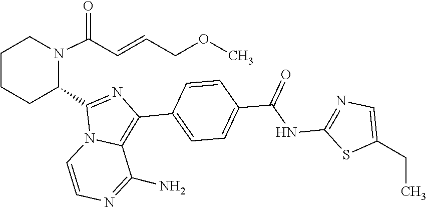 [SMILES: CCc1cnc(NC(=O)c2ccc(-c3nc([C@@H]4CCCCN4C(=O)/C=C/COC)n4ccnc(N)c34)cc2)s1] |
| 46. | (S)-4-(3-(1-Acryloylpiperidin-2-yl)-8- aminoimidazo[1,5-a] pyrazin-1-yl)-2-fluoro- N-(4-propylpyridin-2-yl)benzamide | 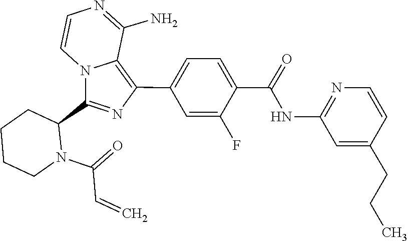 [SMILES: C=CC(=O)N1CCCC[C@H]1c1nc(-c2ccc(C(=O)Nc3cc(CCC)ccn3)c(F)c2)c2c(N)nccn12] |
| 47. | (S, E)-4-(8-Amino-3-(1-(4- (dimethylamino)but-2-enoyl)piperidin-2- yl)imidazo[1,5-a]pyrazin-1-yl)-2-methoxy- N-(4-propylpyridin-2-yl)benzamide | 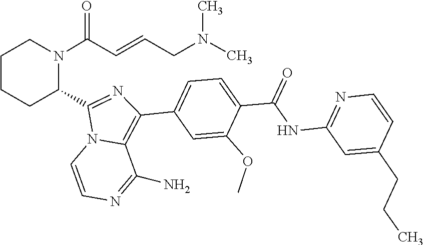 [SMILES: CCCc1ccnc(NC(=O)c2ccc(-c3nc([C@@H]4CCCCN4C(=O)/C=C/CN(C)C)n4ccnc(N)c34)cc2OC)c1] |
| 48. | 4-(8-Amino-3-((S)-1-but-2-ynoylpiperidin-2- yl)imidazo[1,5-a] pyrazin-1-yl)-3-methyl-N- (pyridin-2-yl)benzamide | 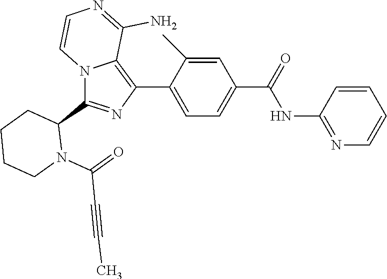 [SMILES: CC#CC(=O)N1CCCC[C@H]1c1nc(-c2ccc(C(=O)Nc3ccccn3)cc2C)c2c(N)nccn12] |
| 49. | 4-(3-(Acrylamidomethyl)-8- aminoimidazo[1,5-a] pyrazin-1-yl)-N- (pyridin-2-yl)benzamide | 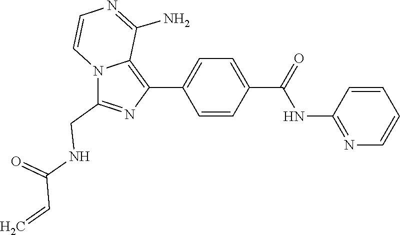 [SMILES: C=CC(=O)NCc1nc(-c2ccc(C(=O)Nc3ccccn3)cc2)c2c(N)nccn12] |
| 50. | (S)-4-(8-Amino-3-(1-but-2- ynamidoethyl)imidazo[1,5-a]pyrazin-1-yl)- N-(pyridin-2-yl)benzamide | 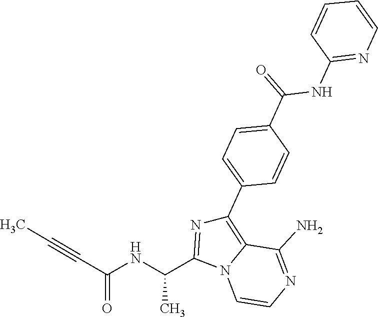 [SMILES: CC#CC(=O)N[C@@H](C)c1nc(-c2ccc(C(=O)Nc3ccccn3)cc2)c2c(N)nccn12] |
| 51. | (S)-S-(2-(2-(8-amino-1-(4-(pyridin-2- ylcarbamoyl)phenyl)imidazo[1,5-a]pyrazin- 3-yl)pyrrolidin-1-yl)-2-oxoethyl) ethanethioate | 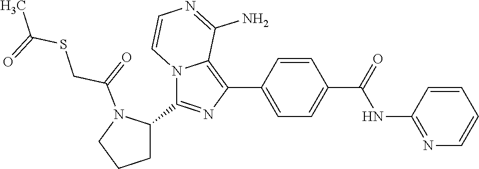 [SMILES: CC(=O)SCC(=O)N1CCC[C@H]1c1nc(-c2ccc(C(=O)Nc3ccccn3)cc2)c2c(N)nccn12] |
| 52. | (S)-4-(8-Amino-3-(1-(4-hydroxy-4- methylpent-2-ynoyl)pyrrolidin-2- yl)imidazo[1,5-a]pyrazin-1-yl)-N(pyridin-2- yl)benzamide | 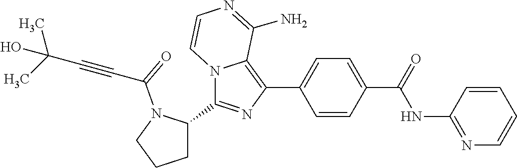 [SMILES: CC(C)(O)C#CC(=O)N1CCC[C@H]1c1nc(-c2ccc(C(=O)Nc3ccccn3)cc2)c2c(N)nccn12] |
| 53. | (S)-4-(8-Amino-3-(1-(6-chloropyrimidine-4- carbonyl)pyrrolidin-2-yl)imidazo[1,5-a] pyrazin-1-yl)-N-(pyridin-2-yl)benzamide | 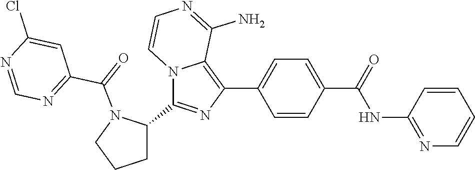 [SMILES: Nc1nccn2c([C@@H]3CCCN3C(=O)c3cc(Cl)ncn3)nc(-c3ccc(C(=O)Nc4ccccn4)cc3)c12] |
| 54. | (S)-4-(8-Amino-3-(1-pent-2-ynoylpyrrolidin- 2-yl)imidazo[1,5-a] pyrazin-1-yl)-N-(pyridin- 2-yl)benzamide | 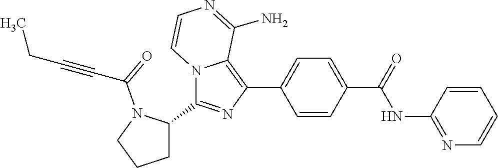 [SMILES: CCC#CC(=O)N1CCC[C@H]1c1nc(-c2ccc(C(=O)Nc3ccccn3)cc2)c2c(N)nccn12] |
| 55. | (S)-4-(8-Amino-3-(1-(3- cyclopropylpropioloyl)pyrrolidin-2- yl)imidazo[1,5-a] pyrazin-1-yl)-N-(pyridin-2- yl)benzamide | 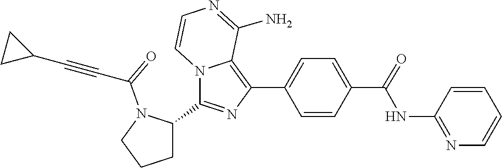 [SMILES: Nc1nccn2c([C@@H]3CCCN3C(=O)C#CC3CC3)nc(-c3ccc(C(=O)Nc4ccccn4)cc3)c12] |
| 56. | (S)-4-(8-Amino-3-(1-hex-2-ynoylpyrrolidin- 2-yl)imidazo[1,5-a] pyrazin-1-yl)-N-(pyridin- 2-yl)benzamide | 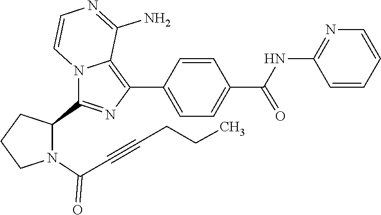 [SMILES: CCCC#CC(=O)N1CCC[C@H]1c1nc(-c2ccc(C(=O)Nc3ccccn3)cc2)c2c(N)nccn12] |
| 57. | 4-(3-(1-Acryloylazepan-2-yl)-8- aminoimidazo[1,5-a]pyrazin-1-yl)-N- (pyridin-2-yl)benzamide | 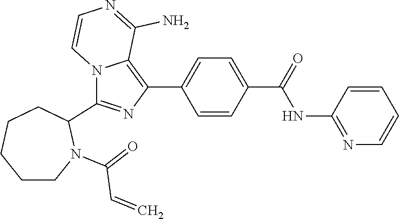 [SMILES: C=CC(=O)N1CCCCCC1c1nc(-c2ccc(C(=O)Nc3ccccn3)cc2)c2c(N)nccn12] |
| 58. | (R)-4-(8-Amino-3-(4-but-2-ynoylmorpholin- 3-yl)imidazo[1,5-a]pyrazin-1-yl)-N-(pyridin- 2-yl)benzamide | 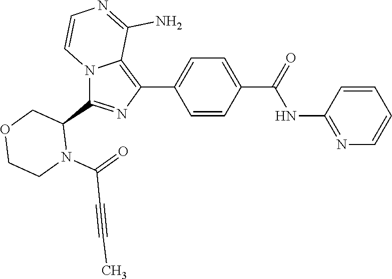 [SMILES: CC#CC(=O)N1CCOC[C@H]1c1nc(-c2ccc(C(=O)Nc3ccccn3)cc2)c2c(N)nccn12] |
| 59. | (S)-4-(8-amino-3-(1-(N-methylbut-2- ynamido)ethyl)imidazo[1,5-a]pyrazin-1-yl)- N-(4-(trifluoromethyl)pyridin-2- yl)benzamide | 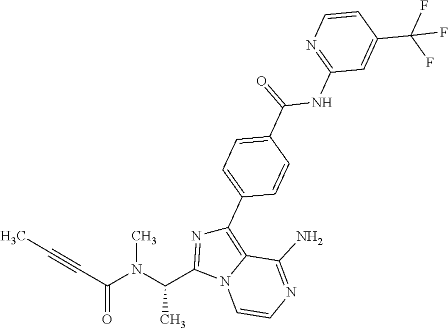 [SMILES: CC#CC(=O)N(C)[C@@H](C)c1nc(-c2ccc(C(=O)Nc3cc(C(F)(F)F)ccn3)cc2)c2c(N)nccn12] |
| 60. | (S)-4-(3-(1-acryloylpyrrolidin-2-yl)-8- aminoimidazo[1,5-a]pyrazin-1-yl)-N-(4- fluoropyridin-2-yl)benzamide | 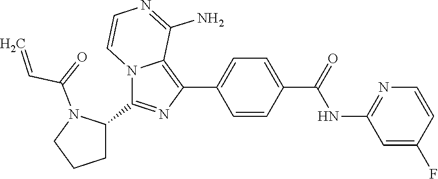 [SMILES: C=CC(=O)N1CCC[C@H]1c1nc(-c2ccc(C(=O)Nc3cc(F)ccn3)cc2)c2c(N)nccn12] |
| 61. | (S)-4-(3-(1-acryloylpyrrolidin-2-yl)-8- aminoimidazo[1,5-a]pyrazin-1-yl)-N-(4- (pyrrolidin-1-yl)pyridin-2-yl)benzamide | 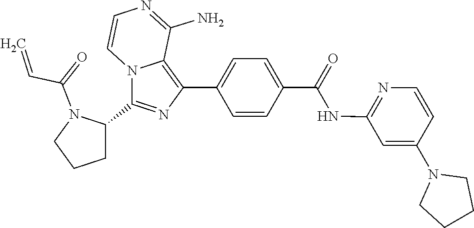 [SMILES: C=CC(=O)N1CCC[C@H]1c1nc(-c2ccc(C(=O)Nc3cc(N4CCCC4)ccn3)cc2)c2c(N)nccn12] |
| 62. | (S)-4-(8-amino-3-(1-but-2-ynoylpiperidin-2- yl)imidazo[1,5-a]pyrazin-1-yl)-N-(4- fluoropyridin-2-yl)benzamide | 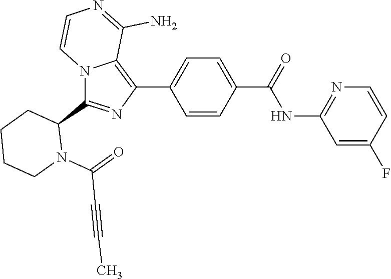 [SMILES: CC#CC(=O)N1CCCC[C@H]1c1nc(-c2ccc(C(=O)Nc3cc(F)ccn3)cc2)c2c(N)nccn12] |
| 63. | (S)-4-(8-amino-3-(1-but-2-ynoylpiperidin-2- yl)imidazo[1,5-a]pyrazin-1-yl)-N-(pyridine- 2-yl)benzamide | 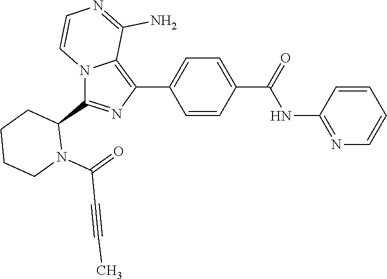 [SMILES: CC#CC(=O)N1CCCC[C@H]1c1nc(-c2ccc(C(=O)Nc3ccccn3)cc2)c2c(N)nccn12] |
| 64. | (S)-4-(3-(1-acryloylpiperidin-2-yl)-8- aminoimidazo[1,5-a]pyrazin-1-yl)-N- (pyridine-2-yl)benzamide | 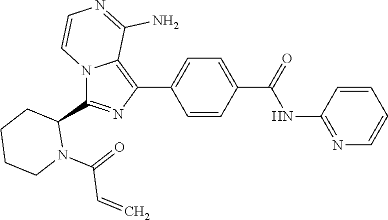 [SMILES: C=CC(=O)N1CCCC[C@H]1c1nc(-c2ccc(C(=O)Nc3ccccn3)cc2)c2c(N)nccn12] |
| 65. | (S)-4-(8-amino-3-(1-but-2-ynoylpyrrolidin- 2-yl)imidazo[1,5-a]pyrazin-1-yl)-N-(4- propylpyridin-2-yl)benzamide | 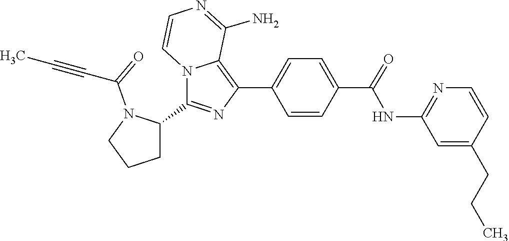 [SMILES: CC#CC(=O)N1CCC[C@H]1c1nc(-c2ccc(C(=O)Nc3cc(CCC)ccn3)cc2)c2c(N)nccn12] |
| 66. | (S,E)-4-(8-amino-3-(1-(4-methoxy-N- methylbut-2-enamido)ethyl)imidazo[1,5- a]pyrazin-1-yl)-N-(4-propylpyridin-2- yl)benzamide | 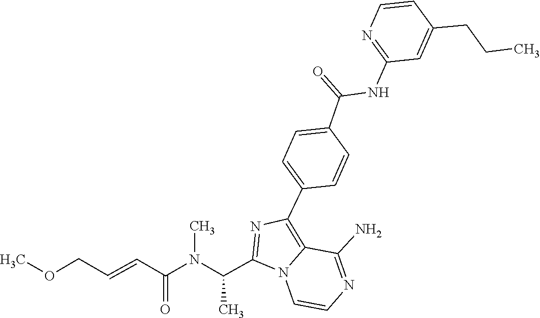 [SMILES: CCCc1ccnc(NC(=O)c2ccc(-c3nc([C@H](C)N(C)C(=O)/C=C/COC)n4ccnc(N)c34)cc2)c1] |
| 67. | (S)-4-(8-amino-3-(1-(vinylsulfonyl)piperidin- 2-yl)imidazo[1,5-a]pyrazin-1-yl)-N-(4- propylpyridin-2-yl)benzamide | 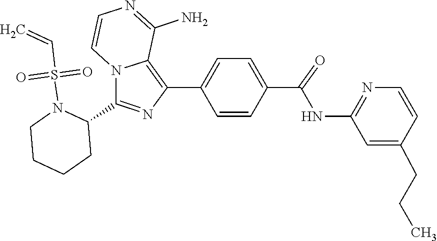 [SMILES: C=CS(=O)(=O)N1CCCC[C@H]1c1nc(-c2ccc(C(=O)Nc3cc(CCC)ccn3)cc2)c2c(N)nccn12] |
| 68. | (S)-4-(3-(1-acryloylpiperidin-2-yl)-8- aminoimidazo[1,5-a]pyrazin-1-yl)-N-(4- propylpyridin-2-yl)benzamide | 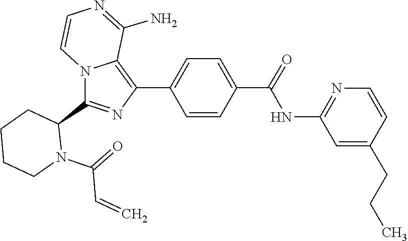 [SMILES: C=CC(=O)N1CCCC[C@H]1c1nc(-c2ccc(C(=O)Nc3cc(CCC)ccn3)cc2)c2c(N)nccn12] |
| 69. | (S)-4-(3-(1-acryloylpyrrolidin-2-yl)-8- aminoimidazo[1,5-a]pyrazin-1-yl)-N-(4- (trifluoromethyl)pyridin-2-yl)benzamide | 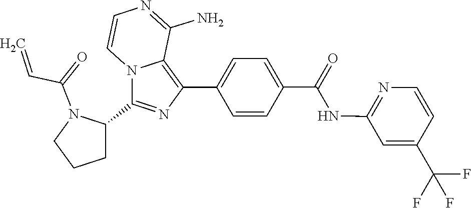 [SMILES: C=CC(=O)N1CCC[C@H]1c1nc(-c2ccc(C(=O)Nc3cc(C(F)(F)F)ccn3)cc2)c2c(N)nccn12] |
| 70. | (S)-4-(8-amino-3-(1-but-2-ynoylpiperidin-2- yl)imidazo[1,5-a]pyrazin-1-yl)-N-(4- (trifluoromethyl)pyridin-2-yl)benzamide | 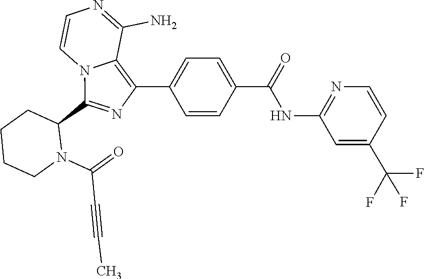 [SMILES: CC#CC(=O)N1CCCC[C@H]1c1nc(-c2ccc(C(=O)Nc3cc(C(F)(F)F)ccn3)cc2)c2c(N)nccn12] |
| 71. | (S)-4-(8-amino-3-(1-but-2-ynoylpiperidin-2- yl)imidazo[1,5-a]pyrazin-1-yl)-N-(4- propylpyridin-2-yl)benzamide | 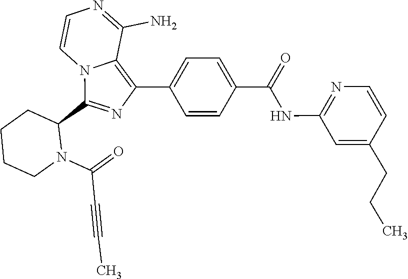 [SMILES: CC#CC(=O)N1CCCC[C@H]1c1nc(-c2ccc(C(=O)Nc3cc(CCC)ccn3)cc2)c2c(N)nccn12] |
| 72. | (S,E)-4-(8-amino-3-(1-(4- (dimethylamino)but-2-enoyl)pyrrolidin-2- yl)imidazo[1,5-a]pyrazin-1-yl)-N-(4- isopropylpyridin-2-yl)benzamide | 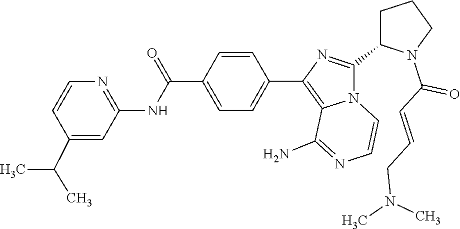 [SMILES: CC(C)c1ccnc(NC(=O)c2ccc(-c3nc([C@@H]4CCCN4C(=O)/C=C/CN(C)C)n4ccnc(N)c34)cc2)c1] |
| 73. | 4-(8-amino-3-((S)-1-(vinylsulfonyl)piperidin- 2-yl)imidazo[1,5-a]pyrazin-1-yl)-3-methyl- N-(pyridin-2-yl)benzamide | 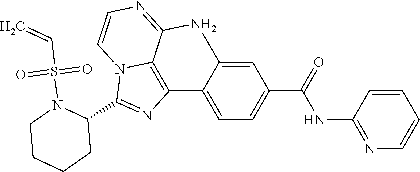 [SMILES: C=CS(=O)(=O)N1CCCC[C@H]1c1nc(-c2ccc(C(=O)Nc3ccccn3)cc2C)c2c(N)nccn12] |
| 74. | (S)-4-(8-amino-3-(1-but-2-ynoylpiperidin-2- yl)imidazo[1,5-a]pyrazin-1-yl)-2-fluoro-N- (4-propylpyridin-2-yl)benzamide | 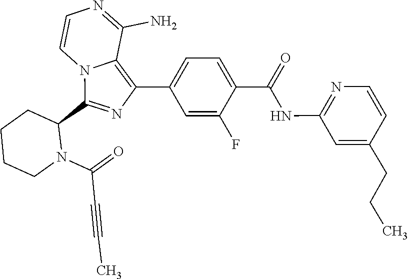 [SMILES: CC#CC(=O)N1CCCC[C@H]1c1nc(-c2ccc(C(=O)Nc3cc(CCC)ccn3)c(F)c2)c2c(N)nccn12] |
| 75. | (S,E)-4-(8-amino-3-(1-(4-methoxy-N- methylbut-2-enamido)ethyl)imidazo[1,5- a]pyrazin-1-yl)-N-(4- (trifluoromethyl)pyridin-2-yl)benzamide | 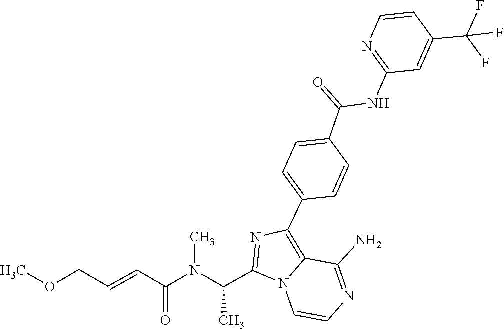 [SMILES: COC/C=C/C(=O)N(C)[C@@H](C)c1nc(-c2ccc(C(=O)Nc3cc(C(F)(F)F)ccn3)cc2)c2c(N)nccn12] |
| 76. | (S,E)-4-(8-amino-3-(1-(4-(dimethylamino)- N-methylbut-2-enamido)ethyl)imidazo[1,5- a]pyrazin-1-yl)-N-(4-propylpyridin-2- yl)benzamide | 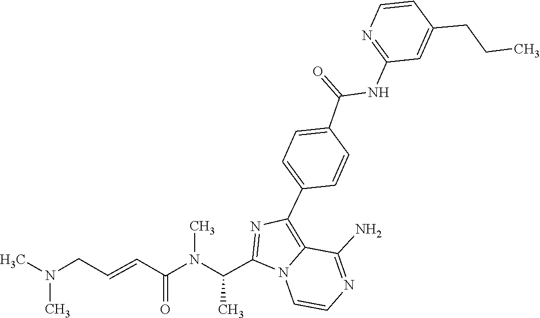 [SMILES: CCCc1ccnc(NC(=O)c2ccc(-c3nc([C@H](C)N(C)C(=O)/C=C/CN(C)C)n4ccnc(N)c34)cc2)c1] |
| 77. | (S,E)-4-(8-amino-3-(1-(4-(pyrrolidin-1- yl)but-2-enoyl)pyrrolidin-2-yl)imidazo[1,5- a]pyrazin-1-yl)-N-(4-propylpyridin-2- yl)benzamide | 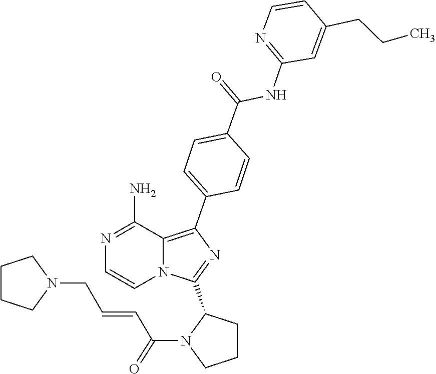 [SMILES: CCCc1ccnc(NC(=O)c2ccc(-c3nc([C@@H]4CCCN4C(=O)/C=C/CN4CCCC4)n4ccnc(N)c34)cc2)c1] |
| 78. | (S,E)-4-(8-amino-3-(1-(4- (dimethylamino)but-2-enoyl)piperidin-2- yl)imidazo[1,5-a]pyrazin-1-yl)-N-(pyridin-2- yl)benzamide | 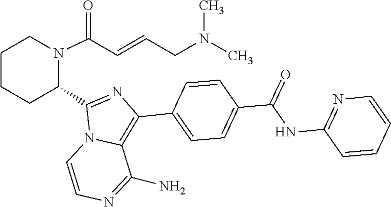 [SMILES: CN(C)C/C=C/C(=O)N1CCCC[C@H]1c1nc(-c2ccc(C(=O)Nc3ccccn3)cc2)c2c(N)nccn12] |
| 79. | (S)-4-(8-amino-3-(1-(2-chloropyrimidine-4- carbonyl)pyrrolidin-2-yl)imidazo[1,5- a]pyrazin-1-yl)-N-(4-propylpyridin-2- yl)benzamide | 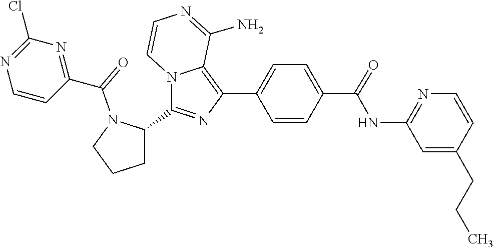 [SMILES: CCCc1ccnc(NC(=O)c2ccc(-c3nc([C@@H]4CCCN4C(=O)c4ccnc(Cl)n4)n4ccnc(N)c34)cc2)c1] |
| 80. | (S)-4-(3-(1-acrylamidoethyl)-8- aminoimidazo[1,5-a]pyrazin-1-yl)-N- (pyridin-2-yl)benzamide | 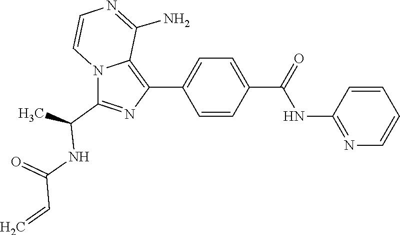 [SMILES: C=CC(=O)N[C@@H](C)c1nc(-c2ccc(C(=O)Nc3ccccn3)cc2)c2c(N)nccn12] |
| 81. | (S)-4-(3-(1-acryloylpyrrolidin-2-yl)-8- aminoimidazo[1,5-a]pyrazin-1-yl)-N- (thiazol-2-yl)benzamide | 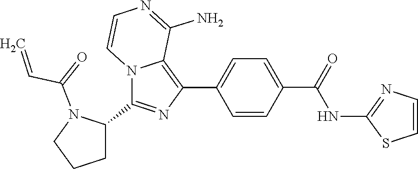 [SMILES: C=CC(=O)N1CCC[C@H]1c1nc(-c2ccc(C(=O)Nc3nccs3)cc2)c2c(N)nccn12] |
| 82. | (S)-4-(8-amino-3-(1-but-2-ynoylpyrrolidin- 2-yl)imidazo[1,5- a]pyrazin-1-yl)-N-(4- isopropylpyridin-2-yl)benzamide | 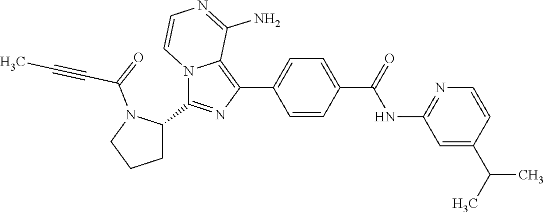 [SMILES: CC#CC(=O)N1CCC[C@H]1c1nc(-c2ccc(C(=O)Nc3cc(C(C)C)ccn3)cc2)c2c(N)nccn12] |
| 83. | (S)-4-(8-amino-3-(1-(2-chloropyrimidine-4- carbonyl)piperidin-2-yl)imidazo[1,5- a]pyrazin-1-yl)-N-(4-propylpyridin-2- yl)benzamide | 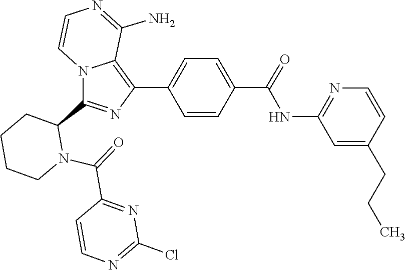 [SMILES: CCCc1ccnc(NC(=O)c2ccc(-c3nc([C@@H]4CCCCN4C(=O)c4ccnc(Cl)n4)n4ccnc(N)c34)cc2)c1] |
| 84. | (S,E)-4-(8-amino-3-(1-(4-methoxybut-2- enoyl)piperidin-2-yl)imidazo[1,5-a]pyrazin- 1-yl)-N-(4-(trifluoromethyl)pyridin-2-yl) benzamide | 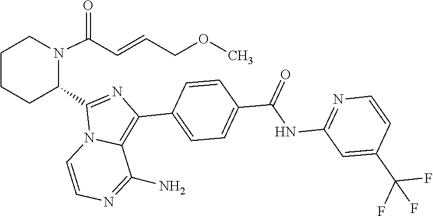 [SMILES: COC/C=C/C(=O)N1CCCC[C@H]1c1nc(-c2ccc(C(=O)Nc3cc(C(F)(F)F)ccn3)cc2)c2c(N)nccn12] |
| 85. | (S)-4-(3-(1-acryloylpiperidin-2-yl)-8- aminoimidazo[1,5-a]pyrazin-1-yl)-N-(4- (trifluoromethyl)pyridin-2-yl)benzamide | 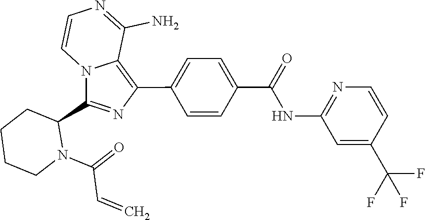 [SMILES: C=CC(=O)N1CCCC[C@H]1c1nc(-c2ccc(C(=O)Nc3cc(C(F)(F)F)ccn3)cc2)c2c(N)nccn12] |
| 86. | (S)-4-(8-amino-3-(1-but-2-ynoylpiperidin-2- yl)imidazo[1,5-a]pyrazin-1-yl)-2-methoxy- N-(4-propylpyridin-2-yl)benzamide | 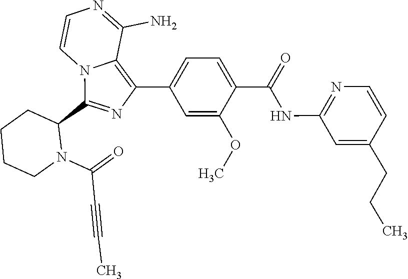 [SMILES: CC#CC(=O)N1CCCC[C@H]1c1nc(-c2ccc(C(=O)Nc3cc(CCC)ccn3)c(OC)c2)c2c(N)nccn12] |
| 87. | (S,E)-4-(8-amino-3-(1-(4-methoxybut-2- enoyl)piperidin-2-yl)imidazo[1,5-a]pyrazin- 1-yl)-2-methoxy-N-(4-propylpyridin-2-yl) benzamide | 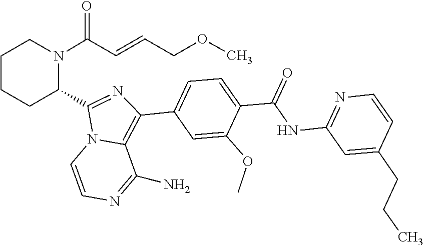 [SMILES: CCCc1ccnc(NC(=O)c2ccc(-c3nc([C@@H]4CCCCN4C(=O)/C=C/COC)n4ccnc(N)c34)cc2OC)c1] |
| 88. | (S)-4-(8-amino-3-(1-(2-chloropyrimidine-4- carbonyl)piperidin-2-yl)imidazo[1,5- a]pyrazin-1-yl)-N-(4- (trifluoromethyl)pyridin-2-yl)benzamide | 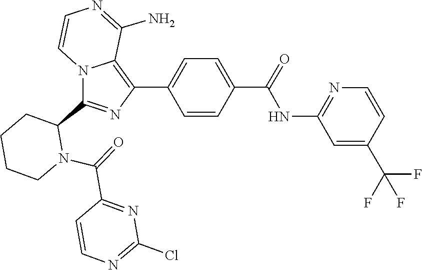 [SMILES: Nc1nccn2c([C@@H]3CCCCN3C(=O)c3ccnc(Cl)n3)nc(-c3ccc(C(=O)Nc4cc(C(F)(F)F)ccn4)cc3)c12] |
| 89. | (S)-4-(8-amino-3-(1-but-2-ynoylpiperidin-2- yl)imidazo[1,5-a]pyrazin-1-yl)-N-(5- ethylthiazol-2-yl)benzamide | 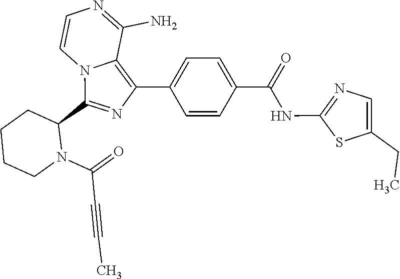 [SMILES: CC#CC(=O)N1CCCC[C@H]1c1nc(-c2ccc(C(=O)Nc3ncc(CC)s3)cc2)c2c(N)nccn12] |
| 90. | (S)-4-(3-(1-acryloylpiperidin-2-yl)-8- aminoimidazo[1,5-a]pyrazin-1-yl)-N-(5- ethylthiazol-2-yl)benzamide | 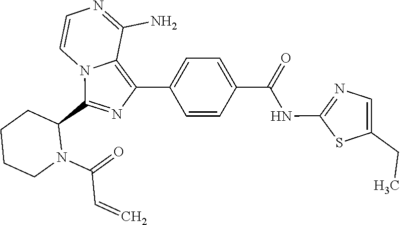 [SMILES: C=CC(=O)N1CCCC[C@H]1c1nc(-c2ccc(C(=O)Nc3ncc(CC)s3)cc2)c2c(N)nccn12] |
| 91. | (S)-4-(8-amino-3-(1-(2-chloropyrimidine-4- carbonyl)piperidin-2-yl)imidazo[1,5- a]pyrazin-1-yl)-N-(5-ethylthiazol-2- yl)benzamide | 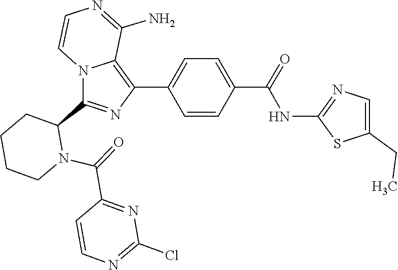 [SMILES: CCc1cnc(NC(=O)c2ccc(-c3nc([C@@H]4CCCCN4C(=O)c4ccnc(Cl)n4)n4ccnc(N)c34)cc2)s1] |
| 92. | (R,E)-4-(8-amino-3-(4-(4-methoxybut-2- enoyl)morpholin-3-yl)imidazo[1,5- a]pyrazin-1-yl)-N-(pyridin-2-yl)benzamide | 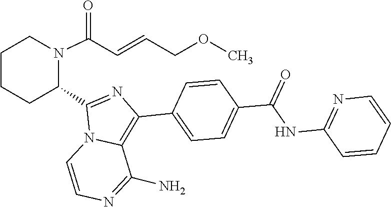 [SMILES: COC/C=C/C(=O)N1CCCC[C@H]1c1nc(-c2ccc(C(=O)Nc3ccccn3)cc2)c2c(N)nccn12] |
| 93. | (S,E)-4-(8-amino-3-(1-(4-methoxybut-2- enoyl)piperidin-2-yl)imidazo[1,5-a]pyrazin- 1-yl)-N-(4-propylpyridin-2-yl)benzamide | 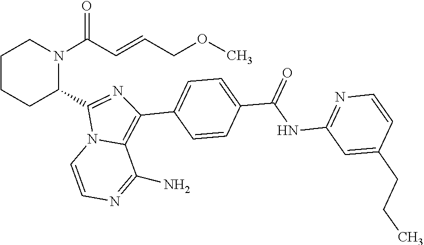 [SMILES: CCCc1ccnc(NC(=O)c2ccc(-c3nc([C@@H]4CCCCN4C(=O)/C=C/COC)n4ccnc(N)c34)cc2)c1] |
| 94. | (S)-4-(3-(1-acryloylpyrrolidin-2-yl)-8- aminoimidazo[1,5-a]pyrazin-1-yl)-N-(4- cyanopyridin-2-yl)benzamide | 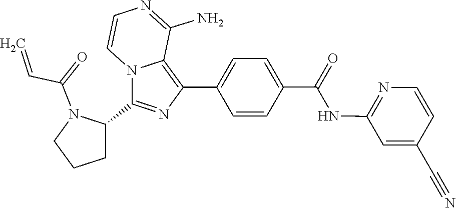 [SMILES: C=CC(=O)N1CCC[C@H]1c1nc(-c2ccc(C(=O)Nc3cc(C#N)ccn3)cc2)c2c(N)nccn12] |
| 95. | (S)-4-(8-amino-3-(1-but-2-ynoylpyrrolidin- 2-yl)imidazo[1,5-a]pyrazin-1-yl)-N-(4- methoxypyridin-2-yl)benzamide | 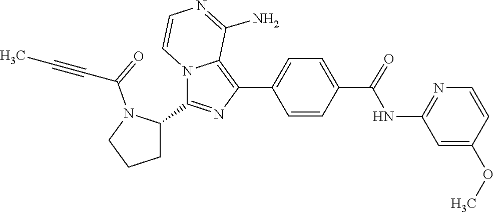 [SMILES: CC#CC(=O)N1CCC[C@H]1c1nc(-c2ccc(C(=O)Nc3cc(OC)ccn3)cc2)c2c(N)nccn12] |
| 96. | (S)-4-(3-(1-acryloylpyrrolidin-2-yl)-8- aminoimidazo[1,5-a]pyrazin-1-yl)-N-(4- methylpyridin-2-yl)benzamide | 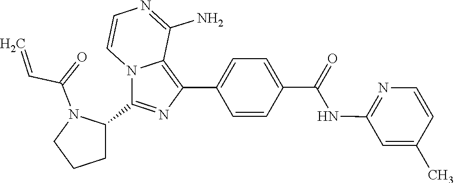 [SMILES: C=CC(=O)N1CCC[C@H]1c1nc(-c2ccc(C(=O)Nc3cc(C)ccn3)cc2)c2c(N)nccn12] |
| 97. | (S)-4-(3-(1-acryloylpyrrolidin-2-yl)-8- aminoimidazo[1,5-a]pyrazin-1-yl)-N-(4- propylpyridin-2-yl)benzamide | 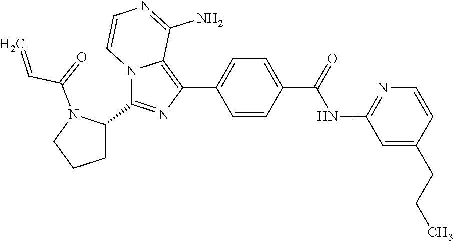 [SMILES: C=CC(=O)N1CCC[C@H]1c1nc(-c2ccc(C(=O)Nc3cc(CCC)ccn3)cc2)c2c(N)nccn12] |
| 98. | (S)-4-(3-(1-acryloylpyrrolidin-2-yl)-8- aminoimidazo[1,5-a]pyrazin-1-yl)-N-(4- ethylpyridin-2-yl)benzamide | 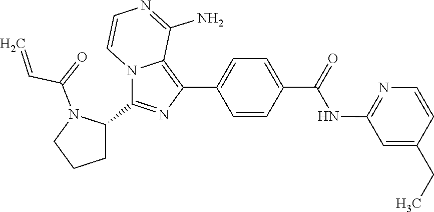 [SMILES: C=CC(=O)N1CCC[C@H]1c1nc(-c2ccc(C(=O)Nc3cc(CC)ccn3)cc2)c2c(N)nccn12] |
| 99. | (S,E)-4-(8-amino-3-(1-(4- (dimethylamino)but-2-enoyl)pyrrolidin-2- yl)imidazo[1,5-a]pyrazin-1-yl)-N-(pyridin-2- yl)benzamide | 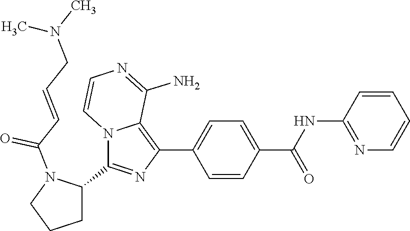 [SMILES: CN(C)C/C=C/C(=O)N1CCC[C@H]1c1nc(-c2ccc(C(=O)Nc3ccccn3)cc2)c2c(N)nccn12] |
| 100. | (S,E)-4-(8-amino-3-(1-(4-methoxybut-2- enoyl)pyrrolidin-2-yl)imidazo[1,5-a]pyrazin- 1-yl)-N-(4-(trifluoromethyl)pyridin-2- yl)benzamide | 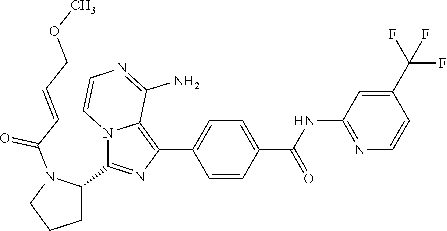 [SMILES: COC/C=C/C(=O)N1CCC[C@H]1c1nc(-c2ccc(C(=O)Nc3cc(C(F)(F)F)ccn3)cc2)c2c(N)nccn12] |
| 101. | (S)-4-(8-amino-3-(1-(2-chloropyrimidine-4- carbonyl)pyrrolidin-2-yl)imidazo[1,5- a]pyrazin-1-yl)-N-(4-methylpyridin-2- yl)benzamide | 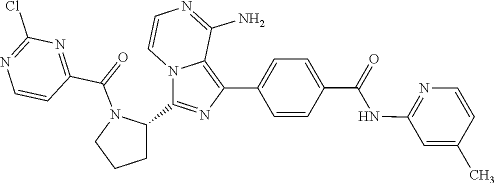 [SMILES: Cc1ccnc(NC(=O)c2ccc(-c3nc([C@@H]4CCCN4C(=O)c4ccnc(Cl)n4)n4ccnc(N)c34)cc2)c1] |
| 102. | (S)-4-(8-amino-3-(1-but-2-ynoylpyrrolidin- 2-yl)imidazo[1,5-a] pyrazin-1-yl)-N-(4- cyanopyridin-2-yl)benzamide | 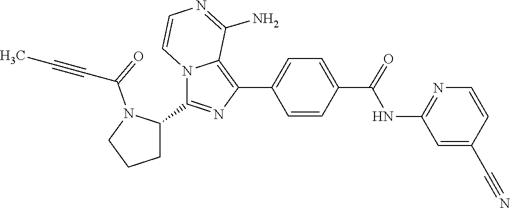 [SMILES: CC#CC(=O)N1CCC[C@H]1c1nc(-c2ccc(C(=O)Nc3cc(C#N)ccn3)cc2)c2c(N)nccn12] |
| 103. | (S)-4-(8-amino-3-(1-but-2- ynoylpyrrolidin- 2-yl)imidazo[1,5-a]pyrazin-1-yl)-N-(4- ethylpyridin-2-yl)benzamide | 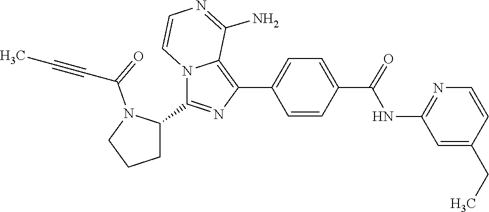 [SMILES: CC#CC(=O)N1CCC[C@H]1c1nc(-c2ccc(C(=O)Nc3cc(CC)ccn3)cc2)c2c(N)nccn12] |
| 104. | (S)-4-(8-amino-3-(1-but-2-ynoylpyrrolidin- 2-yl)imidazo[1,5-a]pyrazin-1-yl)-N-(4- phenylpyridin-2-yl)benzamide | 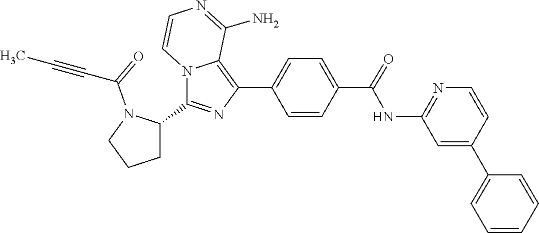 [SMILES: CC#CC(=O)N1CCC[C@H]1c1nc(-c2ccc(C(=O)Nc3cc(-c4ccccc4)ccn3)cc2)c2c(N)nccn12] |
| 105. | (S)-4-(3-(1-acryloylpyrrolidin-2-yl)-8- aminoimidazo[1,5-a]pyrazin-1-yl)N-(4- phenylpyridin-2-yl)benzamide | 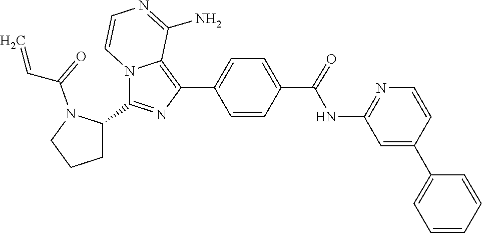 [SMILES: C=CC(=O)N1CCC[C@H]1c1nc(-c2ccc(C(=O)Nc3cc(-c4ccccc4)ccn3)cc2)c2c(N)nccn12] |
| 106. | (R,E)-1-(3-(4-amino-3-(4-phenoxyphenyl)- 1H-pyrazolo[3,4-d]pyrimidin-1-yl)piperidin- 1-yl)-4-(dimethylamino)but-2-en-1-one | 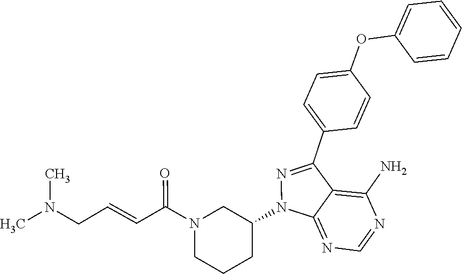 [SMILES: CN(C)C/C=C/C(=O)N1CCC[C@@H](n2nc(-c3ccc(Oc4ccccc4)cc3)c3c(N)ncnc32)C1] |
| 107. | (E)-1-(3-(4-amino-3-(4-phenoxyphenyl)-1H- pyrazolo[3,4-d]pyrimidin-1-yl)piperidin-1- yl)-4-morpholinobut-2-en-1-one | 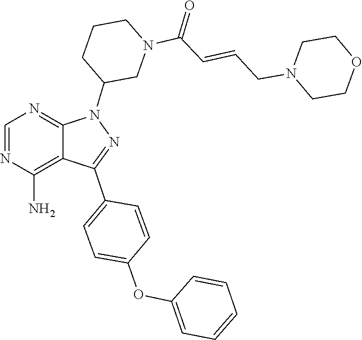 [SMILES: Nc1ncnc2c1c(-c1ccc(Oc3ccccc3)cc1)nn2C1CCCN(C(=O)/C=C/CN2CCOCC2)C1] |
| 108. | 1-(4-(4-amino-3-(4-phenoxyphenyl)-1H- pyrazolo[3,4-d]pyrimidin-1-yl)piperidin-1- yl)prop-2-en-1-one | 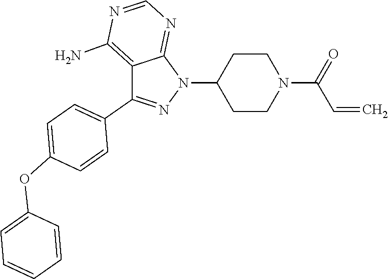 [SMILES: C=CC(=O)N1CCC(n2nc(-c3ccc(Oc4ccccc4)cc3)c3c(N)ncnc32)CC1] |
| 109. | (E)-1-(4-(4-amino-3-(4-phenoxyphenyl)-1H- pyrazolo[3,4-d]pyrimidin-1-yl)piperidin-1- yl)-4-(dimethylamino)but-2-en-1-one | 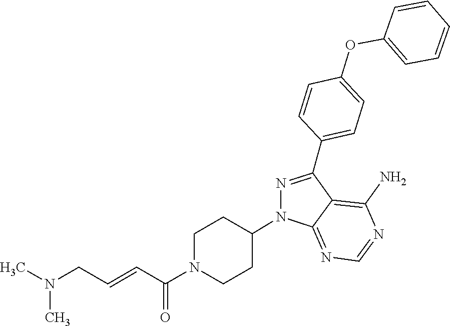 [SMILES: CN(C)C/C=C/C(=O)N1CCC(n2nc(-c3ccc(Oc4ccccc4)cc3)c3c(N)ncnc32)CC1] |
| 110. | (E)-N-((1s,4s)-4-(4-amino-3-(4- phenoxyphenyl)-1H-pyrazolo[3,4- d]pyrimidin-1-yl)cyclohexyl)-4- (dimethylamino)but-2-enamide | 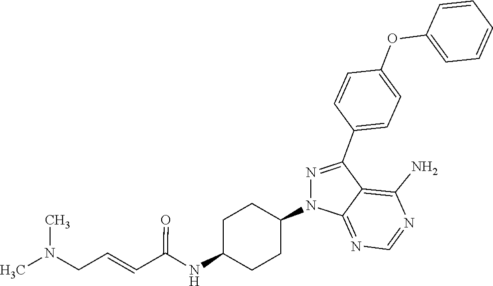 [SMILES: CN(C)C/C=C/C(=O)N[C@H]1CC[C@@H](n2nc(-c3ccc(Oc4ccccc4)cc3)c3c(N)ncnc32)CC1] |
| 111. | 1-(4-(4-amino-3-(4-phenoxyphenyl)-1H- pyrazolo[3,4-d]pyrimidin-1-yl)piperidin-1- yl)prop-2-en-1-one | 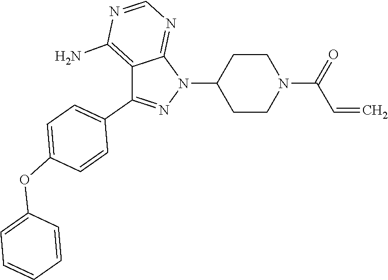 [SMILES: C=CC(=O)N1CCC(n2nc(-c3ccc(Oc4ccccc4)cc3)c3c(N)ncnc32)CC1] |
| 112. | N-((1r,4r)-4-(4-amino-3-(4-phenoxyphenyl)- 1H-pyrazolo[3,4-d]pyrimidin-1- yl)cyclohexyl)acrylamide | 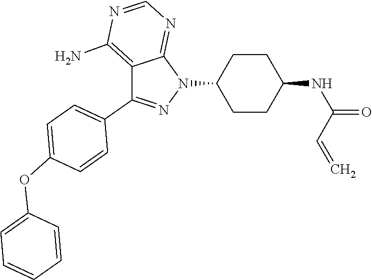 [SMILES: C=CC(=O)N[C@H]1CC[C@H](n2nc(-c3ccc(Oc4ccccc4)cc3)c3c(N)ncnc32)CC1] |
| 113. | (E)-1-((R)-2-((4-amino-3-(4- phenoxyphenyl)-1H-pyrazolo[3,4- d]pyrimidin-1-yl)methyl)pyrrolidin-1-yl)-4- (dimethylamino)but-2-en-1-one | 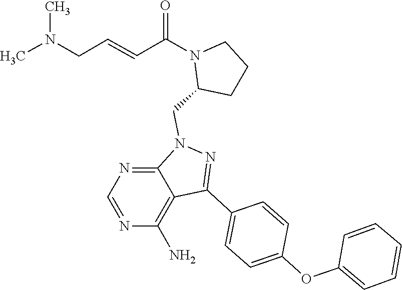 [SMILES: CN(C)C/C=C/C(=O)N1CCC[C@@H]1Cn1nc(-c2ccc(Oc3ccccc3)cc2)c2c(N)ncnc21] |
| 114. | (E)-1-((S)-2-((4-amino-3-(4-phenoxyphenyl)- 1H-pyrazolo[3,4-d]pyrimidin-1- yl)methyl)pyrrolidin-1-yl)-4- (dimethylamino)but-2-en-1-one | 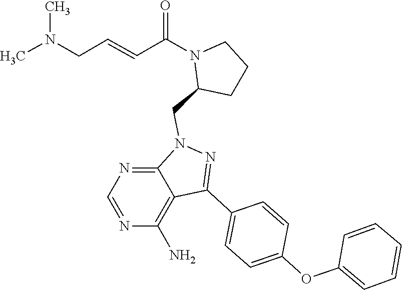 [SMILES: CN(C)C/C=C/C(=O)N1CCC[C@H]1Cn1nc(-c2ccc(Oc3ccccc3)cc2)c2c(N)ncnc21] |
| 115. | 1-((R)-2-((4-amino-3-(4-phenoxyphenyl)- 1H-pyrazolo[3,4-d]pyrimidin-1- yl)methyl)pyrrolidin-1-yl)prop-2-en-1-one | 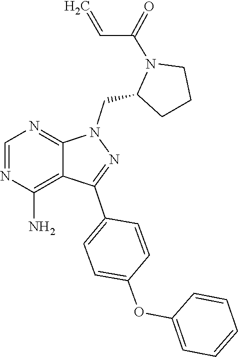 [SMILES: C=CC(=O)N1CCC[C@@H]1Cn1nc(-c2ccc(Oc3ccccc3)cc2)c2c(N)ncnc21] |
| 116. | 1-((S)-2-((4-amino-3-(4-phenoxyphenyl)- 1H-pyrazolo[3,4-d]pyrimidin-1- yl)methyl)pyrrolidin-1-yl)prop-2-en-1-one | 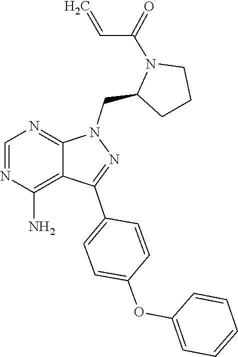 [SMILES: C=CC(=O)N1CCC[C@H]1Cn1nc(-c2ccc(Oc3ccccc3)cc2)c2c(N)ncnc21] |
| 117. | 1-((R)-2-((4-amino-3-(4-phenoxyphenyl)- 1H-pyrazolo[3,4-d]pyrimidin-1- yl)methyl)pyrrolidin-1-yl)but-2-yn-1-one | 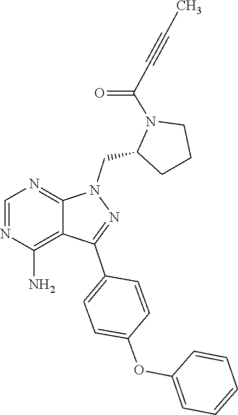 [SMILES: CC#CC(=O)N1CCC[C@@H]1Cn1nc(-c2ccc(Oc3ccccc3)cc2)c2c(N)ncnc21] |
| 118. | 1-((S)-2-((4-amino-3-(4-phenoxyphenyl)-1H- pyrazolo[3,4-d]pyrimidin-1- yl)methyl)pyrrolidin-1-yl)but-2-yn-1-one | 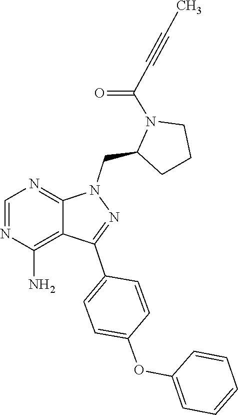 [SMILES: CC#CC(=O)N1CCC[C@H]1Cn1nc(-c2ccc(Oc3ccccc3)cc2)c2c(N)ncnc21] |
| 119. | 1-((R)-3-(4-amino-3-(4-phenoxyphenyl)-1H- pyrazolo[3,4-d]pyrimidin-1-yl)piperidin-1- yl)but-2-yn-1-one | 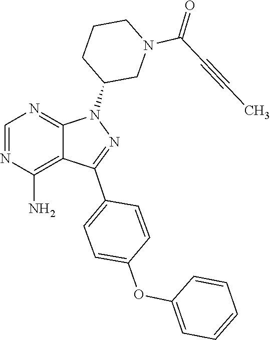 [SMILES: CC#CC(=O)N1CCC[C@@H](n2nc(-c3ccc(Oc4ccccc4)cc3)c3c(N)ncnc32)C1] |
| 120. | (E)-N-((1r,4r)-4-(4-amino-3-(4- phenoxyphenyl)-1H-pyrazolo[3,4- d]pyrimidin-1-yl)cyclohexyl-4- (dimethylamino))but-2-enamide | 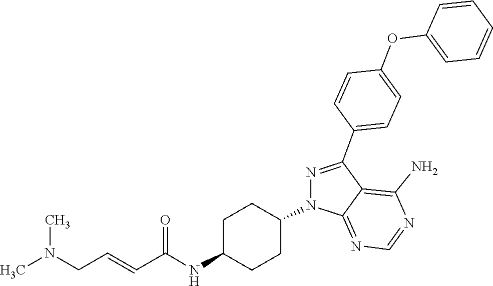 [SMILES: CN(C)C/C=C/C(=O)N[C@H]1CC[C@H](n2nc(-c3ccc(Oc4ccccc4)cc3)c3c(N)ncnc32)CC1] |
| 121. | N-(2-(4-amino-3-(4-phenoxyphenyl)-1H- pyrazolo[3,4-d]pyrimidin-1-yl)ethyl)-N- methylacrylamide | 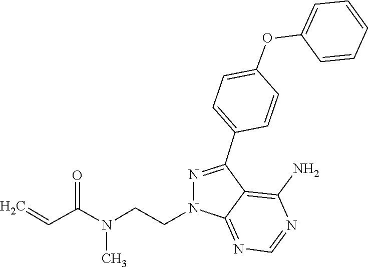 [SMILES: C=CC(=O)N(C)CCn1nc(-c2ccc(Oc3ccccc3)cc2)c2c(N)ncnc21] |
| 122. | (E)-1-(4-(4-amino-3-(4-phenoxyphenyl)-1H- pyrazolo[3,4-d]pyrimidin-1-yl)-4- morpholino)but-2-en-1-one | 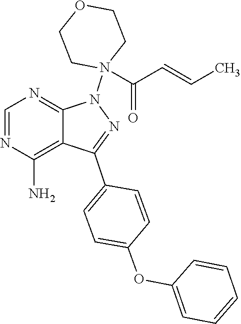 |
| 123. | (E)-1-(2-((S)-2-((4-amino-3-(4- phenoxyphenyl)-1H-pyrazolo[3,4- d]pyrimidin-1-yl)methyl)pyrrolidin-1- yl)morpholino)but-2-en-1-one | 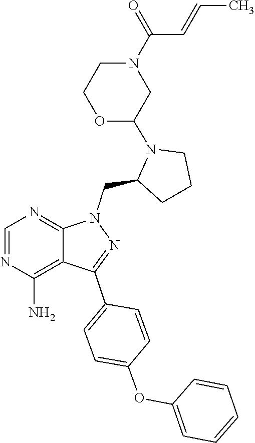 [SMILES: C/C=C/C(=O)N1CCOC(N2CCC[C@H]2Cn2nc(-c3ccc(Oc4ccccc4)cc3)c3c(N)ncnc32)C1] |
| 124. | N-((1s,4s)-4-(4-amino-3-(4-phenoxyphenyl)- 1H-pyrazolo[3,4-d]pyrimidin-1- yl)cyclohexyl)but-2-ynamide | 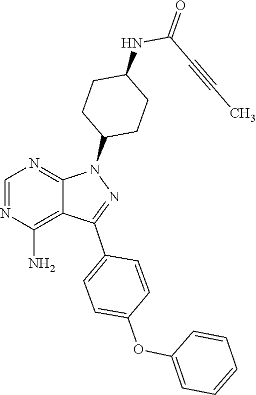 |
| 125. | N-(2-(4-amino-3-(4-phenoxyphenyl)-1H- pyrazolo[3,4-d]pyrimidin-1- yl)ethyl)acrylamide | 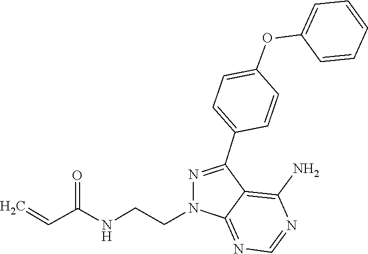 [SMILES: C=CC(=O)NCCn1nc(-c2ccc(Oc3ccccc3)cc2)c2c(N)ncnc21] |
| 126. | (E)-1-((R)-3-(4-amino-3-(4-phenoxyphenyl)- 1H-pyrazolo[3,4-d]pyrimidin-1-yl)piperidin- 1-yl)-4-morpholinobut-2-en-1-one | 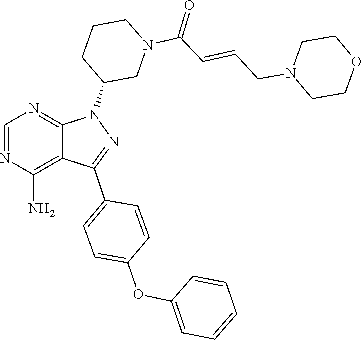 [SMILES: Nc1ncnc2c1c(-c1ccc(Oc3ccccc3)cc1)nn2[C@@H]1CCCN(C(=O)/C=C/CN2CCOCC2)C1] |
| 127. | (E)-N-((1s,4s)-4-(4-amino-3-(4- phenoxyphenyl)-1H-pyrazolo[3,4- d]pyrimidin-1-yl)cyclohexyl)-4- morpholinobut-2-enamide | 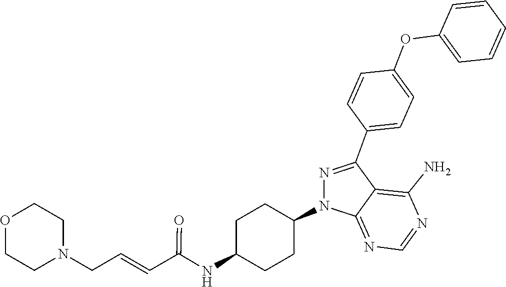 [SMILES: Nc1ncnc2c1c(-c1ccc(Oc3ccccc3)cc1)nn2[C@H]1CC[C@@H](NC(=O)/C=C/CN2CCOCC2)CC1] |
| 128. | 1-(4-(((6-amino-5-(4- phenoxyphenyl)pyrimidin-4- yl)amino)methyl)-4-fluoropiperidin-1- yl)prop-2-en-1-one | 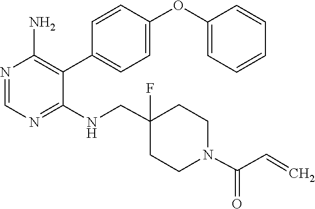 [SMILES: C=CC(=O)N1CCC(F)(CNc2ncnc(N)c2-c2ccc(Oc3ccccc3)cc2)CC1] |
| 129. | N-[3-[[5-fluoro-2-[4-(2- methoxyethoxy)anilino]pyrimidin-4- yl]amino]phenyl]prop-2-enamide | 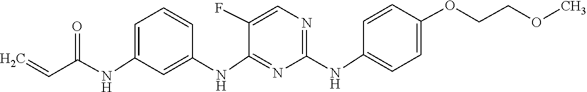 [SMILES: C=CC(=O)Nc1cccc(Nc2nc(Nc3ccc(OCCOC)cc3)ncc2F)c1] |
| 130. | 6-amino-9-[(3R)-1-but-2-ynoylpyrrolidin-3- yl]-7-(4-phenoxyphenyl)purin-8-one | 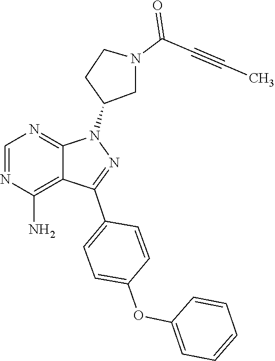 [SMILES: CC#CC(=O)N1CC[C@@H](n2nc(-c3ccc(Oc4ccccc4)cc3)c3c(N)ncnc32)C1] |
| 131. | (7S)-2-(4-phenoxyphenyl)-7-(1-prop-2- enoylpiperidin-4-yl)-4,5,6,7- tetrahydropyrazolo[1,5-a]pyrimidine-3- carboxamide | 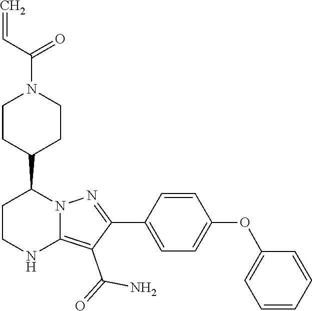 [SMILES: C=CC(=O)N1CCC([C@@H]2CCNc3c(C(N)=O)c(-c4ccc(Oc5ccccc5)cc4)nn32)CC1] |
| 132. | Orelabrutinib (2-(4-phenoxyphenyl)-6-(1- prop-2-enoylpiperidin-4-yl)pyridine-3- carboxamide) | 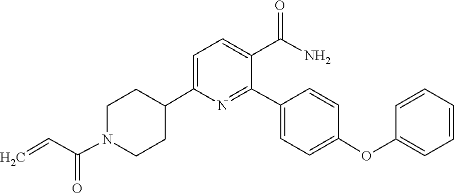 [SMILES: C=CC(=O)N1CCC(c2ccc(C(N)=O)c(-c3ccc(Oc4ccccc4)cc3)n2)CC1] |
| 133. | Remibrutinib (N-[3-[6-amino-5-[2- [methyl(prop-2- enoyl)amino]ethoxy]pyrimidin-4-yl]-5- fluoro-2-methylphenyl]-4-cyclopropyl-2- fluorobenzamide) | 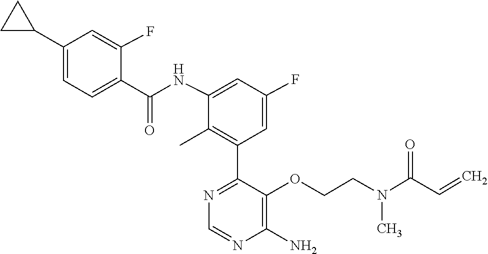 [SMILES: C=CC(=O)N(C)CCOc1c(N)ncnc1-c1cc(F)cc(NC(=O)c2ccc(C3CC3)cc2F)c1C] |
| 134. | Loxo-305 (1H-Pyrazole-4-carboxamide, 5- amino-3-[4-[[(5-fluoro-2- methoxybenzoyl)amino]methyl]phenyl]-1- [(1S)-2,2,2-trifluoro-1-methylethyl]) | 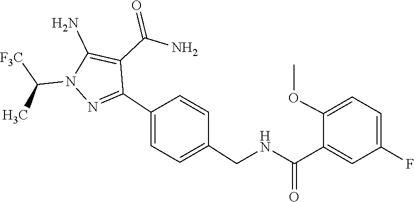 [SMILES: COc1ccc(F)cc1C(=O)NCc1ccc(-c2nn([C@@H](C)C(F)(F)F)c(N)c2C(N)=O)cc1] |
| 135. | TG-1701 (4-amino-1-[(3R)-1-but-2- ynoylpyrrolidin-3-yl]-3-[4-(2,6- difluorophenoxy)phenyl]-6H-pyrrolo[2,3- d]pyridazin-7-one) | 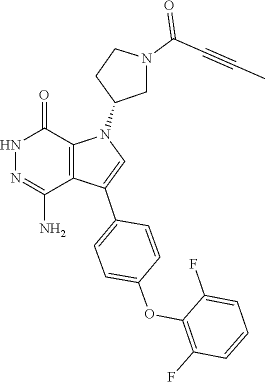 [SMILES: CC#CC(=O)N1CC[C@@H](n2cc(-c3ccc(Oc4c(F)cccc4F)cc3)c3c(N)n[nH]c(=O)c32)C1] |
| 136. | N-((1s,4s)-4-(4-amino-3-(4-phenoxyphenyl)- 1H-pyrazolo[3,4-d]pyrimidin-1- yl)cyclohexyl)acrylamide | 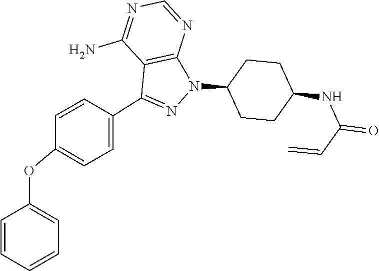 [SMILES: C=CC(=O)N[C@H]1CC[C@@H](n2nc(-c3ccc(Oc4ccccc4)cc3)c3c(N)ncnc32)CC1] |
| 137. | N-((1r,4r)-4-(4-amino-3-(4-phenoxyphenyl)- 1H-pyrazolo[3,4-d]pyrimidin-1- yl)cyclohexyl)but-2-ynamide | 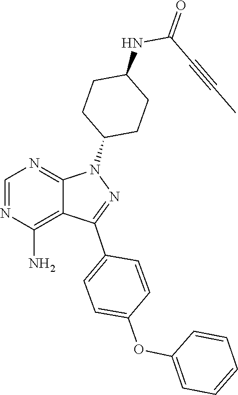 [SMILES: CC#CC(=O)N[C@H]1CC[C@H](n2nc(-c3ccc(Oc4ccccc4)cc3)c3c(N)ncnc32)CC1] |
| 138. | (E)-N-((1r,4r)-4-(4-amino-3-(4- phenoxyphenyl)-1H-pyrazolo[3,4- d]pyrimidin-1-yl)cyclohexyl)-4- morpholinobut-2-enamide | 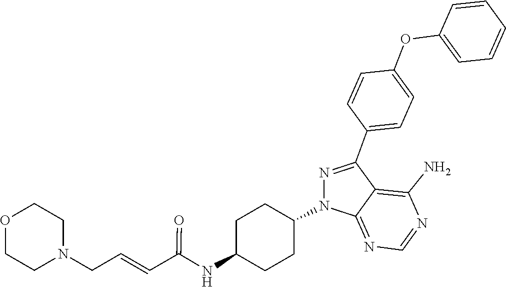 [SMILES: Nc1ncnc2c1c(-c1ccc(Oc3ccccc3)cc1)nn2[C@H]1CC[C@H](NC(=O)/C=C/CN2CCOCC2)CC1] |
| 139. | 2-[3-[2-amino-6-[1-(oxetan-3-yl)-3,6- dihydro-2H-pyridin-4-yl]-7H-pyrrolo[2,3- d]pyrimidin-4-yl]-2- (hydroxymethyl)phenyl]-6-cyclopropyl-8- fluoroisoquinolin-1-one | 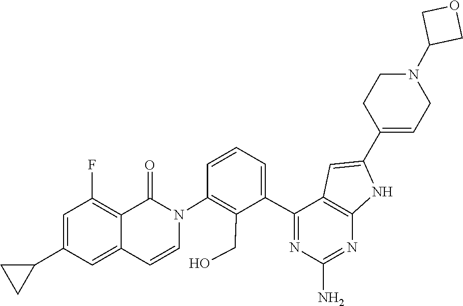 [SMILES: Nc1nc(-c2cccc(-n3ccc4cc(C5CC5)cc(F)c4c3=O)c2CO)c2cc(C3=CCN(C4COC4)CC3)[nH]c2n1] |

In an embodiment, the BTK inhibitor is a BTK-targeted proteolysis targeting chimera (PROTAC) (Arthur (2020) Explor Target Antitumor Ther. 1, 131-152). In an embodiment, the BTK-targeted PROTAC comprises a BTK inhibitor moiety covalently coupled through a linker moiety to an ubiquitin protein ligase (E3) ligase-recruiting moiety. In an embodiment, the BTK inhibitor moiety comprises a BTK inhibitor selected from the group consisting of the compounds listed in Table 2 or pharmaceutically-acceptable salt thereof. In an embodiment, the BTK inhibitor moiety is derived from a BTK inhibitor selected from the group consisting of the compounds listed in Table 2 or pharmaceutically-acceptable salt thereof. In an embodiment, the linker comprises polyethylene glycol (PEG). In an embodiment, the linker 9 to 14 atoms in length, 10 to 12 atoms in length, or 11 atoms in length. In an embodiment, the E3 ligase-recruiting moiety comprises pomalidomide. In an embodiment, the E3 ligase-recruiting moiety is derived from pomalidomide. In an embodiment, the E3 ligase-recruiting moiety comprises lenalidomide. In an embodiment, the E3 ligase-recruiting moiety is derived from lenalidomide. In an embodiment, the E3 ligase-recruiting moiety comprises RG-71120. In an embodiment, the E3 ligase-recruiting moiety is derived from RG-71120. In an embodiment, the E3 ligase-recruiting moiety targets cereblon (CRBN). In an embodiment, the E3 ligase-recruiting moiety targets murine double-minute 2 (MDM2). In an embodiment, the E3 ligase-recruiting moiety targets Von Hippel-Landau (VHL). In an embodiment, the E3 ligase-recruiting moiety targets inhibitor of apoptosis protein (IAP).

In an embodiment, the BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically-acceptable salt thereof.

### Pharmaceutical Compositions

In some embodiments, the invention provides pharmaceutical compositions and uses for treating myeloproliferative disorders. The pharmaceutical compositions are typically formulated to provide a therapeutically effective amount of a BTK inhibitor as the active ingredient, or a pharmaceutically acceptable salt, ester, prodrug, solvate, hydrate or derivative thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other agent(s) may be mixed into a preparation or both components may be formulated into separate preparations for use in combination separately or at the same time.

In some embodiments, the concentration of the BTK inhibitors provided in the pharmaceutical compositions and methods disclosed herein is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the whole pharmaceutical composition or dosage form.

In some embodiments, the concentration of BTK inhibitors provided in the pharmaceutical compositions and methods disclosed herein is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the whole pharmaceutical composition or dosage form.

In some embodiments, the concentration of the BTK inhibitor in the compositions and methods disclosed herein is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v of the whole pharmaceutical composition or dosage form.

In some embodiments, the concentration of the BTK inhibitor of the invention is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v of the whole pharmaceutical composition or dosage form.

In some embodiments, the dose or amount of the BTK inhibitor of the invention is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g present as the active ingredient in the whole pharmaceutical composition or dosage form.

In some embodiments, the dose or amount of the BTK inhibitor of the invention is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g present as the active ingredient in the whole pharmaceutical composition or dosage form.

The BTK inhibitor according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the amount of BTK resynthesis in the human subject in any particular tissue compartment, and also route of administration, the form in which the compound is administered, the gender and age of the subject to be treated, the body weight of the subject to be treated, and the preference and experience of the attending physician.

Efficacy of the compounds and combinations of compounds described herein in treating, preventing and/or managing the indicated diseases or disorders can be tested using various animal or human models known in the art.

### BTK Occupancy and Resynthesis

BTK occupancy (or BTK target occupancy) measures the amount of BTK enzyme that has been covalently bound to a BTK inhibitor at the active site of this kinase protein. Methods for the measurement of BTK occupancy in myeloid cell lysates include, for example, use of selective probe molecules linked to biotin or other probes that may used for detection in various assay platforms. In case of biotin-labeled occupancy probes, the target occupancy can be measured by streptavidin pull down methods that bind the biotinylated probe molecule and BTK protein with standard enzyme-linked immunosorbent assay (ELISA) methods using streptavidin coated plates, as described in Evans, J. Pharmacol. Exp. Ther. 2013, 346:219-228, or following capture using antibodies against BTK with detection using streptavidin conjugated to enzymes or probes for detection. When an appropriately standardized method is used, the BTK occupancy may be reported as a percentage of available BTK that is covalently bound, with 100% occupancy indicating that all BTK is covalently bound. BTK occupancy may also be reported as free BTK per mass of total protein (e.g. pg free BTK/μg total protein or ng free BTK/μg total protein), or may be reported as the percentage of free BTK that is available for detection by the BTK active site probe.

In an embodiment, the invention provides a method of treating a myeloproliferative disorder comprising the step of administering a covalent BTK inhibitor at a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the invention provides a method of treating a myeloproliferative disorder, wherein the disorder is splenomegaly, comprising the step of administering a BTK inhibitor at a dose effective to obtain a BTK occupancy selected from the group consisting of greater than 90%, greater than 91%, greater than 92%, greater than 93%, greater than 94%, greater than 95%, greater than 96%, greater than 97%, greater than 98%, and greater than 99%.

In an embodiment, the invention provides a method of treating a myeloproliferative disorder comprising the step of administering a BTK inhibitor at a dose effective to obtain an average BTK occupancy selected from the group consisting of 80%, 81%, 82%, 83%, 84%, 85%, 86%, 87%, 88%, 89%, 90%, 91%, 92%, 93%, 94%, 95%, 96%, 97%, 98%, 99%, 99.5%, and 100%. In an embodiment, the invention provides a method of treating a myeloproliferative disorder, wherein the disorder is primary myelofibrosis, post-polycythemia vera myelofibrosis, post-essential thrombocythemia myelofibrosis, or splenomegaly comprising the step of administering a BTK inhibitor at a dose effective to obtain an average BTK occupancy selected from the group consisting of about 80%, about 85%, about 90%, about 95%, about 96%, about 97%, about 98%, and about 99%.

BTK resynthesis refers to the process by which new BTK enzyme is produced after existing BTK enzyme becomes occupied by covalent attachment to a BTK inhibitor. This can occur within a viable cell over time, or can occur during the generation of new cells upon proliferation or transit from a tissue compartment of therapeutic interest (e.g. spleen) into the assayed compartment (e.g. blood). The BTK resynthesis rate can be measured by determining BTK occupancy over a period of time in a specific compartment (e.g. blood); or by determining the presence of free BTK (not bound to drug) in a specimen sampled from a compartment of interest at a certain time after administration of a fully occupying dose of a covalent BTK inhibitor. The BTK resynthesis rate can also be obtained as an average rate.

BTK resynthesis rate may be determined by fitting BTK inhibitor occupancy data against a suitable biochemical kinetics model. For example, if the target occupancy assay determines free protein, and assuming full occupancy of the protein after dosing, free BTK may be determined after dosing by applying a pharmacokinetic-pharmacodynamic (PK/PD) model that estimates the t_{1/2} of BTK target occupancy as a function of the BTK resynthesis rate. Another approach is to use the following equation: free BTK=new BTK/h*h (where h refers to hours), or apply a linear extrapolation from data observing the decline in BTK target occupancy and the return of BTK signaling function during the washout period after dosing with a BTK inhibitor. The latter approaches assumes a linear synthesis rate for BTK over time, whereas the former approach is more nuanced and predicts a first or second order terminal elimination phase for BTK target occupancy for BTK target occupancy following the concentration-time profile of the active BTK inhibitor as measured in plasma. The target occupancy assay may be performed such that the free BTK is not an absolute value but is based on 100% free BTK in the individual prior to dosing. A value of 100% occupancy of BTK is determined by incubation of the same test sample with a high saturating dose of an exogenous covalent BTK inhibitor. The BTK resynthesis rate (new BTK/hour) may be expressed as percent per hour, as a percentage of predose free BTK. Alternately, if the expression of BTK protein is quantified, the BTK resynthesis rate may be expressed quantitatively, as pg/μg tissue/hour or pg/μg total protein/hour.

In an embodiment, the invention includes a method of treating myeloproliferative disorder which exhibits a rate of BTK resynthesis, which can be measured in sites of disease (e.g. spleen) using specific imaging agents to detect the presence of unoccupied BTK target sites when combined with CT scans, positron emission tomography (PET) imaging, magnetic resonance imaging (MRI), or near infrared fluorescence imaging, or other in vivo imaging modalities, to customize the treatment of a specific disease based on the regeneration rate of BTK in diseased tissue. In an embodiment, the PET probe is a ¹¹C-labeled BTK inhibitor. In an embodiment, the PET probe is a ¹¹C-labeled BTK inhibitor. In an embodiment, the PET probe is a ¹¹C-labeled BTK inhibitor, such as the BTK inhibitors set forth in Table 1, labeled at a specific carbon position, such as an exocyclic carbon position, which may be prepared by synthetic methods known to those of ordinary skill in the art. In an embodiment, the PET probe is a ¹⁸F-labeled BTK inhibitor, such as any of the BTK inhibitors in Table 1, wherein a hydrogen is substituted by a ¹⁸F nucleus, such as an substitution at an aryl position, which may be prepared by synthetic methods known to those of ordinary skill in the art. Preparation of organic molecules containing ¹¹C, ¹⁸F, ¹³N, and ¹⁵O labels for PET imaging is described, e.g., in Miller, Angewandte Chemie Int. Ed., 2008, 47, 8998-9033.

In an embodiment, the BTK resynthesis rate in myeloid cells is selected from the group consisting of about 0.1 pg free BTK/μg total protein/hour, about 0.5 pg free BTK/μg total protein/hour, about 1 pg free BTK/μg total protein/hour, about 2 pg free BTK/μg total protein/hour, about 3 pg free BTK/μg total protein/hour, about 4 pg free BTK/μg total protein/hour, about 5 pg free BTK/μg total protein/hour, about 6 pg free BTK/μg total protein/hour, about 7 pg free BTK/μg total protein/hour, about 8 pg free BTK/μg total protein/hour, about 9 pg free BTK/μg total protein/hour, about 10 pg free BTK/μg total protein/hour, about 20 pg free BTK/μg total protein/hour, and about 50 pg free BTK/μg total protein/hour.

In an embodiment, the BTK resynthesis half-life in myeloid cells is selected from the group consisting of 2 hours, 4 hours, 6 hours, 8 hours, 10 hours, 12 hours, 14 hours, 16 hours, 18 hours, 20hours, 22 hours, 24 hours, 48 hours, and 72 hours.

### Dosing Regimens

The amount of the BTK inhibitor administered will be dependent on the human subject being treated, the severity of the disorder or condition, the rate of administration, the disposition of the compounds and the discretion of the prescribing physician. For each disease setting and for subsets of patients within each disease setting, the resynthesis rate of BTK in target cells/tissues of interest, and the desired percentage of inhibition of BTK function, will also influence the amount of the BTK inhibitor administered. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, by dividing such larger doses into several small doses for administration throughout the day. In some embodiments the dose is a high saturating dose sufficient to bind to all available BTK, including newly expressed BTK.

In selected embodiments, the BTK inhibitor is administered in single or multiple doses. Dosing may be about once, twice, three times, four times, five times, six times, or more than six times per day. Dosing may be about once a month, once every two weeks, once a week, or once every other day. In other embodiments, the BTK inhibitor is administered about once per day to about 6 times per day. In another embodiment the administration of the BTK inhibitor continues for less than about 7 days. In yet another embodiment the administration continues for more than about 6, 10, 14, 28 days, two months, six months, or one year. In some cases, continuous dosing is achieved and maintained as long as necessary.

Administration of the BTK inhibitor may continue as long as necessary. In selected embodiments, the BTK inhibitor is administered for more than 1, 2, 3, 4, 5, 6, 7, 14, or 28 days. In some embodiments, the BTK inhibitor is administered for less than 28, 14, 7, 6, 5, 4, 3, 2, or 1 day. In selected embodiments, the BTK inhibitor is administered on an ongoing basis—e.g., for the treatment of chronic effects.

An effective amount of the inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant. In an embodiment, the BTK inhibitor is administered orally.

The effective amount of a BTK inhibitor may be determined according to an aspect of the present invention by comparing and interpreting the BTK occupancy or BTK resynthesis rate obtained from myeloid cells in different tissue compartments. In an embodiment, the human subject is suffering from primary myelofibrosis, post-polycythemia vera myelofibrosis, post-essential thrombocythemia myelofibrosis, or splenomegaly, wherein the malignant myeloid cells display a difference in BTK occupancy or BTK resynthesis rate between cells in tissue compartments (including spleen, lymph nodes, liver, blood and bone marrow), wherein the BTK occupancy or BTK resynthesis rate is greater in the tissue compartment by an amount selected from the group consisting of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In an embodiment, the human subject is suffering from primary myelofibrosis, post-polycythemia vera myelofibrosis, post-essential thrombocythemia myelofibrosis, or splenomegaly, wherein the malignant myeloid cells display a difference in BTK occupancy or BTK resynthesis rate between malignant myeloid cells in different tissue compartments (including spleen, lymph nodes, liver, bone marrow, and sites of primary or metastatic myelofibrosis, including blood), wherein the BTK occupancy or BTK resynthesis rate is greater in the tissue compartment by an amount in the range selected from the group consisting of 0 to 10%, 10 to 20%, 20% to 30%, 30% to 40%, 40% to 50%, 50% to 60%, 60% to 70%, 70% to 80%, 80% to 90%, or 90% up to 100%.

In an embodiment, the invention includes a method of treating a myeloproliferative disorder that exhibits a higher rate of BTK resynthesis in myeloid cells in tissue compartments, including in the spleen, lymph nodes, liver and bone marrow relative to the BTK resynthesis rate to B cells in the blood, comprising the step of administering a dose of a compound to reduce the rate of BTK resynthesis, wherein the compound is from Table 1, or a pharmaceutically-acceptable salt, cocrystal, hydrate, solvate, or prodrug thereof, wherein the dose is administered once daily, twice daily, or three times daily, and wherein myeloproliferative disorder is primary myelofibrosis, post-polycythemia vera myelofibrosis, post-essential thrombocythemia myelofibrosis, or splenomegaly.

### Methods of Treating Myeloproliferative Disorders

The present invention relates to a method of treating a myeloproliferative disorder or myelofibrosis comprising the step of administering to a human in need thereof a BTK inhibitor compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the invention provides a method of treating a myeloproliferative disorder, wherein the disorder is splenomegaly, comprising the step of administering a BTK inhibitor at a dose effective to obtain a BTK occupancy selected from the group consisting of greater than 90%, greater than 91%, greater than 92%, greater than 93%, greater than 94%, greater than 95%, greater than 96%, greater than 97%, greater than 98%, and greater than 99%. In some embodiments, the MF is primary myelofibrosis, also known as chronic idiopathic myelofibrosis. This is in contrast with myelofibrosis that develops secondary to polycythemia vera or essential thrombocythaemia. In some embodiments, however, the invention encompasses treating myelofibrosis that develops secondary to polycythemia vera or essential thrombocythaemia. In some embodiments, the BTK inhibitor is any of the compounds in Table 1 or a pharmaceutically acceptable salt thereof.

The present invention also relates to a method of treating a myelofibrosis comprising the step of administering to a human in need thereof a BTK inhibitor or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the myelofibrosis is selected from the group consisting of primary myelofibrosis, secondary myelofibrosis, myelofibrosis secondary to polycythemia vera, myelofibrosis secondary to essential thrombocythemia, myelofibrosis secondary to chronic myeloid leukemia, and idiopathic myelofibrosis. In an embodiment, the myelofibrosis is selected from the group consisting of primary myelofibrosis, post-polycythemia vera myelofibrosis, and post-essential thrombocythemia myelofibrosis. In an embodiment, the primary myelofibrosis is selected from the group consisting of prefibrotic/early stage PMF and overt fibrotic stage PMF. In an embodiment, the human is determined as hydroxyurea intolerant (unacceptable side effects). In an embodiment, the human subject is determined as hydroxyurea resistant (inadequate response). In an embodiment, the human subject has splenomegaly. In an embodiment, the human subject has splenomegaly and is phlebotomy-dependent. In an embodiment, the human subject is phlebotomy-dependent without splenomegaly.

In an embodiment, the human subject is JAK2 inhibitor naïve (i.e. has never received therapy with a JAK inhibitor, including inhibitory small molecule drugs such as ruxolitinib noted to have additional activity, such as JAK2 or JAK3 inhibition). In an embodiment, the human subject is JAK2 inhibitor intolerant, e.g., due to becoming RBC transfusion dependent, due to myelosuppression, or due to GI intolerance. In an embodiment, the human subject is JAK2 inhibitor ineligible, e.g., due to a low platelet count. In an embodiment, the human subject has relapsed after JAK2 inhibitor treatment. In an embodiment, the human subject is refractory to JAK2 inhibitor treatment. In an embodiment, the human subject failed ruxolitinib or fedratinib therapy. Failed ruxolitinib, fedratinib or pacritinib therapy includes, but is not limited to, (i) the absence of a reduction in the severity or progression (e.g., on spleen size or symptom burden) of any MPN in a human subject receiving ruxolitinib or fedratinib, or (ii) a relapse of any myelofibrosis in a human subject following a clinical response to ruxolitinib or fedratinib therapy. In an embodiment, failed ruxolitinib or fedratinib therapy is the absence of a reduction in the severity or progression of any myelofibrosis in a human subject receiving ruxolitinib or fedratinib. In an embodiment, failed ruxolitinib or fedratinib therapy is a relapse of any myelofibrosis in a human subject following ruxolitinib or fedratinib therapy. In an embodiment, the BTK inhibitor is the compound selected from Table 1 and pharmaceutically acceptable salts thereof.

In an embodiment, the invention relates to a method of treating primary myelofibrosis in a human that comprises the step of administering to said human a therapeutically effective amount of a BTK inhibitor or a pharmaceutically acceptable salt thereof, wherein the BTK inhibitor is a compound selected from Table 1 in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In some embodiments, the BTK inhibitor is a covalent or irreversible BTK inhibitor.

In an embodiment, the invention relates to a method of treating post-polycythemia vera myelofibrosis in a human that comprises the step of administering to said human a therapeutically effective amount of a BTK inhibitor or a pharmaceutically acceptable salt thereof, wherein the BTK inhibitor is a compound selected from Table 1 in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a method of treating post-essential thrombocythemia myelofibrosis in a human that comprises the step of administering to said human a therapeutically effective amount of a BTK inhibitor or a pharmaceutically acceptable salt thereof, wherein the BTK inhibitor is a compound selected from Table 1 in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the human subject has an accumulation of malignant CD34+ myeloid cells in their hematopoietic tissue (e.g., in their spleen). These malignant CD34+ myeloid cells have decreased expression of CXCR4 relative to normal myeloid cells. In an embodiment, the BTK inhibitor is administered in a therapeutically effective amount sufficient to detach from supportive stromal tumor microenvironment and/or stimulate migration of the malignant CD34+ myeloid cells to peripheral blood from the hematopoietic tissue (e.g., the bone marrow or spleen) of the human subject.

In an embodiment the invention relates to a method of stimulating migration of malignant CD34+ myeloid cells from hematopoietic tissue (e.g., the spleen) to the peripheral blood in a human subject suffering from myelofibrosis, comprising administering a BTK inhibitor to the human subject in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the human subject has an accumulation of malignant CD34+ myeloid cells in their spleen. These malignant CD34+ myeloid cells have decreased expression of CXCR4 relative to normal myeloid cells.

In an embodiment, the present invention relates to a method of treating secondary myelofibrosis comprising the step of administering to a human in need thereof a BTK inhibitor in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the BTK inhibitor is a compound selected from Table 1 or a pharmaceutically acceptable salt thereof, wherein the secondary myelofibrosis is selected from the group consisting of myelofibrosis secondary to polycythemia vera, and myelofibrosis secondary to essential thrombocythemia. In an embodiment, the polycythemia vera is phlebotomy-dependent polycythemia vera. In an embodiment, the human subject is determined as hydroxyurea intolerance (unacceptable side effects). In an embodiment, the human subject is determined as hydroxyurea resistant (inadequate response). In an embodiment, the human subject has splenomegaly. In an embodiment, the human has splenomegaly and is phlebotomy-dependent. In an embodiment, the human subject is phlebotomy-dependent without splenomegaly. In an embodiment, the human subject has failed previous MF therapy with ruxolitinib or fedratinib.

In an embodiment, the BTK inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275mg QD, 300 mg QD, 325 mg QD, 350 mg QD, 360 mg QD, 375 mg QD, 400 mg QD, 425 mg QD, 450 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID. In an embodiment, the BTK inhibitor is administered to a human according to Dosing Regimens section.

In an embodiment, myelofibrosis is selected from primary myelofibrosis, post-polycythemia vera myelofibrosis, and post-essential thrombocythemia myelofibrosis. In an embodiment, the myelofibrosis is selected from primary myelofibrosis, post-polycythemia vera myelofibrosis, and post-essential thrombocythemia myelofibrosis, and the human subject failed ruxolitinib or fedratinib therapy for PMF, post PV-MF or post ET-MF.

In an embodiment, the myelofibrosis is characterized by the presence of a CALR mutation.

In an embodiment, the myelofibrosis is characterized by the presence of an MPL mutation.

In an embodiment, the myelofibrosis is characterized by JAK2V617F mutation in the human subject.

In an embodiment, the myelofibrosis is characterized by one or more mutations selected from the group consisting of JAK (including JAK2V617F), MPL, CALR mutations and combinations thereof. In an embodiment, the mutation is a JAK2 exon 12 somatic mutation. In an embodiment, the myelofibrosis is characterized by a triple negative combination, where mutations in none of JAK2, MPL and CALR are present.

In an embodiment, the invention relates to a method of treating myelofibrosis secondary to polycythemia vera in a human that comprises the step of administering to said human a therapeutically effective amount of a BTK inhibitor or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the BTK inhibitor is a compound selected from Table 1.

In an embodiment, the invention relates to a method of treating myelofibrosis secondary to essential thrombocythemia in a human that comprises the step of administering to said human a therapeutically effective amount of a BTK inhibitor or a pharmaceutically acceptable salt thereof, wherein the BTK inhibitor is a compound selected from Table 1 in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a method of treating myelofibrosis secondary to chronic myeloid leukemia in a human that comprises the step of administering to said human a therapeutically effective amount of a BTK inhibitor or a pharmaceutically acceptable salt thereof, wherein the BTK inhibitor is a compound selected from Table 1 in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a method of treating myelofibrosis in a human that comprises the step of administering to said human a therapeutically effective amount of a BTK inhibitor compound selected from Table 1 or a pharmaceutically acceptable salt thereof, in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99% wherein the dose is selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 400 mg QD, 425 mg QD, 450 mg QD, 480 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID. In an embodiment, the MF is selected from the group consisting of myelofibrosis, primary myelofibrosis, post-polycythemia vera myelofibrosis, and post-essential thrombocythemia myelofibrosis. In an embodiment, the primary myelofibrosis is selected from the group consisting of prefibrotic/early stage PMF and overt fibrotic stage PMF.

In an embodiment, the invention relates to a method of treating myelofibrosis in a human that comprises the step of administering to said human a therapeutically effective amount of a BTK inhibitor compound selected from Table 1 or a pharmaceutically acceptable salt thereof, in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the dose is selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 400 mg QD, 425 mg QD, 450 mg QD, 480 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID, wherein the MF is selected from the group consisting of MF secondary to polycythemia vera, MF secondary to essential thrombocythemia and MF secondary to CML.

In an embodiment, the invention relates to a method of treating primary myelofibrosis in a human that comprises the step of administering to said human a therapeutically effective amount of a BTK inhibitor compound selected from Table 1 or a pharmaceutically acceptable salt thereof, in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the dose is selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 400 mg QD, 425 mg QD, 450 mg QD, 480 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID.

In an embodiment, the invention relates to a method of treating post-polycythemia vera myelofibrosis in a human that comprises the step of administering to said human a therapeutically effective amount of a BTK inhibitor compound selected from Table 1 or a pharmaceutically acceptable salt thereof, in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the dose is selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 400 mg QD, 425 mg QD, 450 mg QD, 480 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID.

In an embodiment, the invention relates to a method of treating post-essential thrombocythemia myelofibrosis in a human that comprises the step of administering to said human a therapeutically effective amount of a BTK inhibitor compound selected from Table 1 or a pharmaceutically acceptable salt thereof, in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the dose is selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 400 mg QD, 425 mg QD, 450 mg QD, 480 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, 400 mg BID, 425 mg BID, 450 mg BID, and 480 mg BID.

In an embodiment, the invention relates to a use of a BTK inhibitor or a pharmaceutically acceptable salt thereof, in the manufacture of a medicament for treating myelofibrosis wherein the treating comprises the step of administering to a human one or more doses of a BTK inhibitor compound from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the MF is selected from the group consisting of primary myelofibrosis, post-polycythemia vera myelofibrosis, and post-essential thrombocythemia myelofibrosis. In an embodiment, the primary myelofibrosis is selected from the group consisting of prefibrotic/early stage PMF and overt fibrotic stage PMF.

In an embodiment, the invention relates to a use of a BTK inhibitor or a pharmaceutically acceptable salt thereof, in the manufacture of a medicament for treating primary myelofibrosis, wherein the treating comprises the step of administering to a human one or more doses of a BTK inhibitor compound from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a BTK inhibitor or a pharmaceutically acceptable salt thereof, in the manufacture of a medicament for treating post-polycythemia vera myelofibrosis, wherein the treating comprises the step of administering to a human one or more doses of a BTK inhibitor compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a BTK inhibitor or a pharmaceutically acceptable salt thereof, in the manufacture of a medicament for treating post-essential thrombocythemia myelofibrosis, wherein the treating comprises the step of administering to a human one or more doses of a BTK inhibitor compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a BTK inhibitor or a pharmaceutically acceptable salt thereof, in the manufacture of a medicament for treating myelofibrosis secondary to polycythemia vera, wherein the treating comprises the step of administering to a human one or more doses of a BTK inhibitor compound from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a BTK inhibitor or a pharmaceutically acceptable salt thereof, in the manufacture of a medicament for treating myelofibrosis secondary to essential thrombocythemia, wherein the treating comprises the step of administering to a human one or more doses of a BTK inhibitor compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a BTK inhibitor or a pharmaceutically acceptable salt thereof, in the manufacture of a medicament for treating myelofibrosis secondary to chronic myeloid leukemia, wherein the treating comprises the step of administering to a human one or more doses of a BTK inhibitor compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%.

In an embodiment, the invention relates to a use of a composition comprising a BTK inhibitor selected from Table 1 or a pharmaceutically acceptable salt thereof, in the manufacture of a medicament for treating myelofibrosis comprising the step of administering to a human one or more doses of the composition comprising the BTK inhibitor or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the myelofibrosis is selected from the group consisting of primary myelofibrosis, post-polycythemia vera myelofibrosis, and post-essential thrombocythemia myelofibrosis. In an embodiment, the primary myelofibrosis is selected from the group consisting of prefibrotic/early stage PMF and overt fibrotic stage PMF.

The methods described above may be used as first-line cancer therapy, or after treatment with conventional therapy, including ruxolitinib, fedratinib or pacritinib.

A BTK inhibitor or a pharmaceutically acceptable salt thereof may also be used in combination with radiation therapy, hormone therapy, surgery and immunotherapy, which therapies are well known to those skilled in the art, for treating myelofibrosis selected from the group consisting of primary myelofibrosis, idiopathic myelofibrosis, post-polycythemia vera myelofibrosis, and post-essential thrombocythemia myelofibrosis. In an embodiment, the primary myelofibrosis is selected from the group consisting of prefibrotic/early stage PMF and overt fibrotic stage PMF.

### Methods of Treating Myeloproliferative Neoplasms

The present invention also relates to a method of treating a MPN comprising the step of administering to a human in need thereof a BTK inhibitor, or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the MPN is selected from the group consisting of polycythemia vera, myelofibrosis, primary myelofibrosis, thrombocythemia, essential thrombocythemia, idiopathic systemic mastocystosis (SM), chronic neutrophilic leukemia (CNL), chronic eosinophilic leukemia-not otherwise specified (CEL-NOS), unclassified myeloproliferative neoplasm (MPN-U), myelodysplastic syndrome (MDS), and systemic mast cell disease (SMCD). In an embodiment, the MPN is selected from the group consisting of chronic neutrophilic leukemia (CNL), chronic eosinophilic leukemia, chronic myelomonocytic leukemia (CMML), atypical chronic myeloid leukemia (aCML), juvenile myelomonocytic leukemia (JMML), hypereosinophilic syndromes (HES), and myelodysplastic/myeloproliferative neoplasms with ring sideroblasts and thrombocytosis (MDS/MPN-RS-T). In an embodiment, the polycythemia vera is phlebotomy-dependent polycythemia vera. In an embodiment, the human is determined as hydroxyurea intolerance (unacceptable side effects). In an embodiment, the human subject is determined as hydroxyurea resistant (inadequate response). In an embodiment, the human subject has splenomegaly. In an embodiment, the human subject has splenomegaly and is phlebotomy-dependent. In an embodiment, the human subject is phlebotomy-dependent without splenomegaly.

In an embodiment, the human subject is JAK2 inhibitor naïve (i.e. has never received therapy with a JAK2 inhibitor). In an embodiment, the human subject is JAK2 inhibitor intolerant, e.g., due to becoming RBC transfusion dependent, or due to GI intolerance. In an embodiment, the human subject is JAK2 inhibitor ineligible, e.g., due to a low platelet count. In an embodiment, the human subject has relapsed after JAK2 inhibitor treatment. In an embodiment, the human subject is refractory to JAK2 inhibitor treatment. In an embodiment, the human subject failed ruxolitinib or fedratinib therapy. Failed ruxolitinib or fedratinib therapy includes, but is not limited to, (i) the absence of a reduction in the severity or progression of any MPN in a human subject receiving ruxolitinib or fedratinib, or (ii) a relapse of any myelofibrosis in a human subject following ruxolitinib or fedratinib therapy. In an embodiment, failed ruxolitinib or fedratinib therapy is the absence of a reduction in the severity or progression of any myelofibrosis symptom in a human subject receiving ruxolitinib or fedratinib. In an embodiment, failed ruxolitinib or fedratinib therapy is a relapse of any myelofibrosis in a human subject following ruxolitinib or fedratinib therapy. In an embodiment, the BTK inhibitor is the compound selected from Table 1 and pharmaceutically acceptable salts thereof.

In an embodiment, the present invention relates to a method of treating a MPN comprising the step of administering to a human in need thereof a BTK inhibitor, wherein the BTK inhibitor is a compound selected from Table 1 or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%, wherein the MPN is selected from the group consisting of polycythemia vera and essential thrombocythemia. In an embodiment, the polycythemia vera is phlebotomy-dependent polycythemia vera. In an embodiment, the human subject is determined as hydroxyurea intolerance (unacceptable side effects). In an embodiment, the human subject is determined as hydroxyurea resistant (inadequate response). In an embodiment, the human subject has splenomegaly. In an embodiment, the human has splenomegaly and is phlebotomy-dependent. In an embodiment, the human subject is phlebotomy-dependent without splenomegaly. In an embodiment, the human subject has failed previous MPN therapy with ruxolitinib or fedratinib.

The present invention also relates to a method of treating a blast phase MPN (MPN-BP) comprising the step of administering to a human in need thereof a BTK inhibitor, or a pharmaceutically acceptable salt thereof in a dose effective to obtain a BTK occupancy selected from the group consisting of greater than 85%, greater than 90%, greater than 91%, greater than 92%, greater than 93%, greater than 94%, greater than 95%, greater than 96%, greater than 97%, greater than 98%, and greater than 99%. In an embodiment, the MPN-BP is selected from the group consisting of blast phase polycythemia vera (BP-PV), blast phase myelofibrosis, blast phase thrombocythemia, blast phase essential thrombocythemia (BP-ET), blast phase systemic mastocystosis (BP-SM), blast phase chronic neutrophilic leukemia (BP-CNL), blast phase myelodysplastic syndrome (BP-MDS), and blast phase systemic mast cell disease (BP-SMCD). In an embodiment, the MPN-BP is selected from the group consisting of blast phase chronic neutrophilic leukemia (BP-CNL), blast phase chronic eosinophilic leukemia, blast phase chronic myelomonocytic leukemia (BP-CMML), blast phase atypical chronic myeloid leukemia (BP-aCML), blast phase juvenile myelomonocytic leukemia (BP-JMML), blast phase hypereosinophilic syndromes (BP-HES), and blast phase myelodysplastic/myeloproliferative neoplasms with ring sideroblasts, thrombocytosis (BP-MDS/MPN-RS-T) and erythroblastic leukemia. In an embodiment, the blast phase polycythemia vera is phlebotomy-dependent polycythemia vera. In an embodiment, the human is determined as hydroxyurea intolerance (unacceptable side effects). In an embodiment, the human subject is determined as hydroxyurea resistant (inadequate response). In an embodiment, the human subject has splenomegaly. In an embodiment, the human subject has splenomegaly and is phlebotomy-dependent. In an embodiment, the human subject is phlebotomy-dependent without splenomegaly.

In an embodiment, the human subject is JAK2 inhibitor naïve (i.e. has never received therapy with a JAK2 inhibitor. In an embodiment, the human subject is JAK2 inhibitor intolerant, e.g., due to becoming RBC transfusion dependent, or due to GI intolerance. In an embodiment, the human subject is JAK2 inhibitor ineligible, e.g., due to a low platelet count. In an embodiment, the human subject has relapsed after JAK2 inhibitor treatment. In an embodiment, the human subject is refractory to JAK2 inhibitor treatment. In an embodiment, the human subject failed ruxolitinib or fedratinib therapy. Failed ruxolitinib or fedratinib therapy includes, but is not limited to, (i) the absence of a reduction in the severity or progression of any MPN in a human subject receiving ruxolitinib or fedratinib, or (ii) a relapse of any myelofibrosis in a human subject following ruxolitinib or fedratinib therapy. In an embodiment, failed ruxolitinib or fedratinib therapy is the absence of a reduction in the severity or progression of any myelofibrosis in a human subject receiving ruxolitinib or fedratinib. In an embodiment, failed ruxolitinib or fedratinib therapy is a relapse of any myelofibrosis in a human subject following ruxolitinib or fedratinib therapy. In an embodiment, the BTK inhibitor is the compound selected from Table 1 and pharmaceutically acceptable salts thereof. In an embodiment, the BTK inhibitor is administered to a human according to Section Dosages and Dosing Regimens.

In an embodiment, the MPN is characterized by CALR mutation.

In an embodiment, the MPN is characterized by MPL mutation.

In an embodiment, the MPN is characterized by JAK2V617F mutation.

In an embodiment, the MPN is characterized by one or more mutations selected from the group consisting of JAK (including JAK2V617F and exon 12 mutations), MPL, CALR and mixtures thereof. In an embodiment, the MPN is characterized by a BCR-ABL mutation.

### EXAMPLES

### Example 1: BTK Resynthesis Rates in Different Cell Types

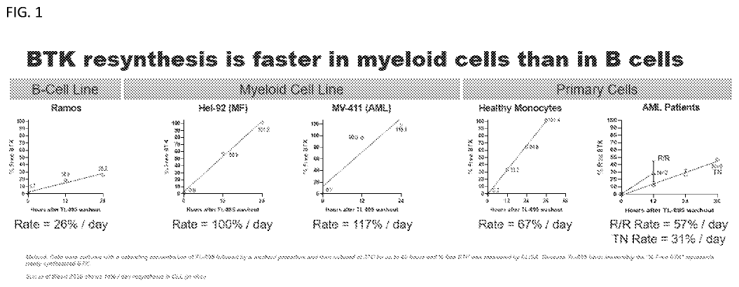

Various cell types and tissues show different rates of BTK synthesis (see FIG. 1). Cells were cultured with a saturating concentration of Compound 128 to occupy all BTK protein (0% free BTK). This was followed by a washout of Compound 128 and culturing for up to 48 hours with no BTK inhibitor to allow the cells to synthesize new BTK protein. The rate of BTK protein synthesis was determined by measuring the free BTK by ELISA at different time points. RAMOS B cells resynthesized BTK at a rate of only 26% per day, while Hel-92 and MV-411 cells resynthesized BTK at a rate of over 100% per day. A culture of primary myeloid cells from a healthy donor resynthesized BTK at a rate of 67% per day while cultures of primary cells from patients with relapsed AML had a BTK resynthesis rate of 57% per day. The rate of BTK resynthesis in naïve AML patients was roughly 30% per day, with low inter-patient variability. These data are consistent with malignant myeloid cells having a higher BTK resynthesis rate than not only B cells, but also normal myeloid cells.

### Example 2: BTK Resynthesis Over Time in Myeloid Cells

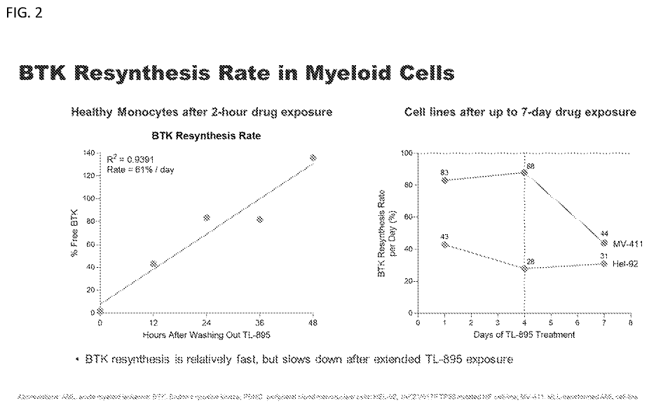

BTK resynthesis in each cell type was measured as described in Example 1. The left graph (see FIG. 2) depicts the BTK resynthesis rate in monocytes from a normal individual following a two hour exposure to Compound 128. The BTK resynthesis rate was determined to be 61% per day in normal monocytes. The right graph (see FIG. 2) depicts the BTK resynthesis rate in the presence of BTK inhibitor Compound 128 in two different cell lines. MV-411 cells (AML) had an initial BTK resynthesis rate of 83% per day but was reduced to 44% per day after continued exposure to Compound 128 for seven days. HEL-92 (myelofibrosis) cells had an initial BTK resynthesis rate of 43% per day but this decreased to as low as 28% per day after repeated exposure to Compound 128. Both cell lines had decreased BTK resynthesis rates after continued exposure to Compound 128. Hel-92 cells demonstrated a decreased BTK resynthesis rate after 4 days of Compound 128 treatment and MV-411 cells demonstrated a decreased BTK resynthesis rate after 7 days of Compound 128 treatment.

In myeloproliferative disorders, the de novo resynthesis rate of BTK following covalent inhibition by Compound 128 is greater than the de novo BTK resynthesis rate in B-cell malignancies. For example, myelofibrotic HEL-92 cells have a four-fold higher de novo BTK resynthesis rate compared to a Ramos B-lymphoblastoid cell line. The short half-life of Compound 128 (1 to 2 hours in vivo), coupled with the faster BTK turnover in myelofibrosis in particular, and myeloproliferative disorders in general, requires higher BID dosing. Thus, a 300 mg BID dose in subjects with myelofibrosis should ensure maximal BTK occupancy at trough levels of this drug.

### Example 3: BTK Resynthesis Over Time in AML and MF Cells

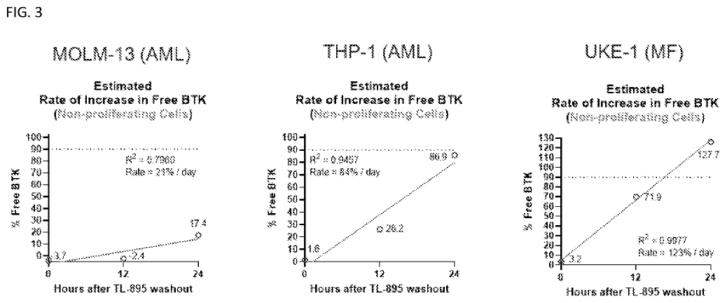

BTK resynthesis was measured in additional AML and MF cell types as described in Example 1 following a one hour exposure to Compound 128 (see FIG. 3). MOLM-13 cells resynthesized BTK at a rate of only 21% per day, while THP-1 cells resynthesized BTK at a rate of 84% per day and UKE-1 cells resynthesized BTK at a rate of over 100% per day.

1. A method of treating a myeloproliferative disorder in a human subject in need thereof comprising:
(a) administering a Bruton's tyrosine kinase (BTK) inhibitor or a pharmaceutically acceptable salt thereof to the human at a first dose for a first period of time sufficient to provide 95% or greater BTK occupancy in a tissue compartment; and
(b) administering the BTK inhibitor or a pharmaceutically acceptable salt thereof to the human at a second dose for a second period of time, wherein the second dose is equal to, or less than the first dose, and is sufficient to maintain the 95% or greater BTK occupancy in the tissue compartment.

2. The method of claim 1, wherein the target BTK occupancy is selected from the group consisting of greater than 96%, greater than 97%, greater than 98%, and greater than 99%.

3. The method of any one of claims 1 and 2, wherein the BTK occupancy is estimated by the BTK resynthesis rate in a tissue compartment containing malignant myeloid cells.

4. The method of any one of claims 1 to 3, wherein the tissue compartment is selected from the group consisting of peripheral blood, bone marrow, lymph node, liver and spleen.

5. The method of any previous claim, wherein the BTK occupancy is evaluated based on the average BTK resynthesis rate in a population of patients with a myeloid cell signaling disorder.

6. The method of any previous claim, further comprising the step of determining the target BTK occupancy in the tissue compartment using a relative resynthesis rate.

7. The method of any previous claim, wherein the BTK inhibitor is selected from Table 1.

8. The method of any previous claim, wherein the BTK inhibitor is

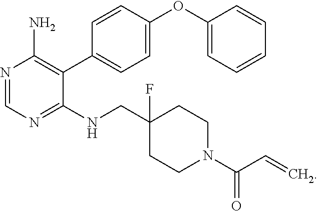 [SMILES: C=CC(=O)N1CCC(F)(CNc2ncnc(N)c2-c2ccc(Oc3ccccc3)cc2)CC1]

9. The method of any previous claim, wherein the BTK inhibitor is administered orally.

10. The method of any previous claim, wherein the first dose is 150 mg of the BTK inhibitor.

11. The method of any previous claim, wherein the second dose is 150 mg of the BTK inhibitor.

12. The method of any one of claims 1 to 11, wherein the myeloproliferative disorder is a mononuclear myeloid cell malignancy.

13. The method of any one of claims 1 to 11, wherein the myeloproliferative disorder is a polymorphonuclear myeloid cell malignancy.

14. The method of any one of claims 1 to 11, wherein the myeloproliferative disorder is primary myelofibrosis, secondary myelofibrosis, myelofibrosis secondary to polycythemia vera, myelofibrosis secondary to essential thrombocythemia, myelofibrosis secondary to chronic myeloid leukemia, or idiopathic myelofibrosis.

15. The method of any previous claim, wherein the BTK inhibitor has a serum half-life of 2.5 hours or less.

16. The method of any one of claims 1 to 15, wherein the first dose of the BTK inhibitor is administered once daily.

17. The method of any one of claims 1 to 15, wherein the first dose of the BTK inhibitor is administered twice daily.

18. The method of any one of claims 1 to 15, wherein the first dose of the BTK inhibitor is administered three times daily.

19. The method of any one of claims 1 to 15, wherein the second dose of the BTK inhibitor is administered once daily.

20. The method of any one of claims 1 to 15, wherein the second dose of the BTK inhibitor is administered twice daily.

21. The method of any one of claims 1 to 15, wherein the second dose of the BTK inhibitor is administered three times daily.

22. The method of any one of claims 1 to 21, wherein the first dose of the BTK inhibitor is selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, and 450 mg.

23. The method of any one of claims 1 to 21, wherein the second dose of the BTK inhibitor is selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, and 450 mg.

24. The method of any one of claims 1 to 21, wherein the first period is selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, and 21 days.

25. The method of any one of claims 1 to 21, wherein the second period is selected from the group consisting of 2 weeks, 1 month, 2 months, 3 months, 6 months, 9 months, and 1 year.
